(19) United States
(12) Patent Application Publication
(10) Pub. No.: US 2026/0327188 A1
(43) Pub. Date: Oct. 22, 2026

(54) HETEROCYCLIC COMPOUNDS

(71) Applicant: Yongshuai CHAI, Shanghai (CN)

(72) Inventors: Yongshuai CHAI, Shanghai (CN); Zhaoyin WANG, Shanghai (CN)

(21) Appl. No.: 19/379,139

(22) Filed: Nov. 4, 2025

Related U.S. Application Data
(60) Provisional application No. 63/834,658, filed on Apr. 11, 2025.
(60) Provisional application No. 63/804,023, filed on May 12, 2025.

(30) Foreign Application Priority Data
Sep. 8, 2025 (CN) 202511279803.X

(51) Int. Cl. C07D 207/09; A61K 31/4025; C07B 59/00

(52) U.S. Cl. CPC C07D 207/09; A61K 31/4025; C07B 59/002

(57) ABSTRACT

The present invention is in the field of novel therapeutics, and specifically relates to a class of heterocyclic compounds as Lp(a) inhibitors, and their pharmaceutically acceptable salts and compositions, which are useful to lower Lp(a) level in patients.

### BACKGROUND

One of the risk factors for cardiovascular disease (CVD) is lipid disorder. There are effective treatment methods to lower Low-density lipoprotein cholesterol (LDL-c) and triglycerides. Elevated lipoprotein(a) (Lp(a)) plasma level is an independent risk factor for CVD. However, elevation of Lp(a) is a challenging target to treat effectively in clinics with few options available for patients due to rather complicated and repeated procedures involving blood filtration to remove Lp(a). The present invention relates to a class of heterocyclic compounds, pharmaceutically acceptable salts thereof, pharmaceutical composition, and the use of these compounds to lower plasma Lp(a).

### DESCRIPTION OF THE INVENTION

The present invention features a compound of Formula IA as Lp(a) inhibitors or its pharmaceutically acceptable salts:

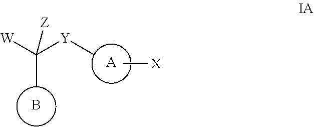

Wherein,
W is CO₂H;
X is selected from the following groups:

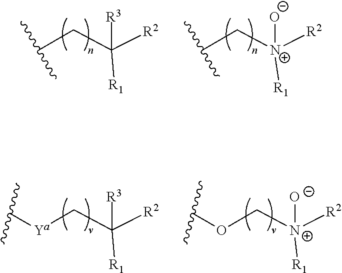

Y^{a} is O, S, CH₂, NH, NC₁₋₆alkyl, NC₃₋₆cycloalkyl, N-phenyl, NC(O)C₁₋₆alkyl, NC(O)C₃₋₆yccloalkyl, NC(O)phenyl; the said alkyl, cycloalkyl and phenyl are unsubstituted or substituted with one or more halogen;
R¹ and R² are independently selected the following moieties:

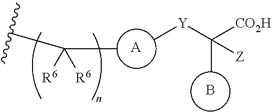

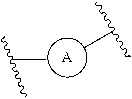

is independently selected from:

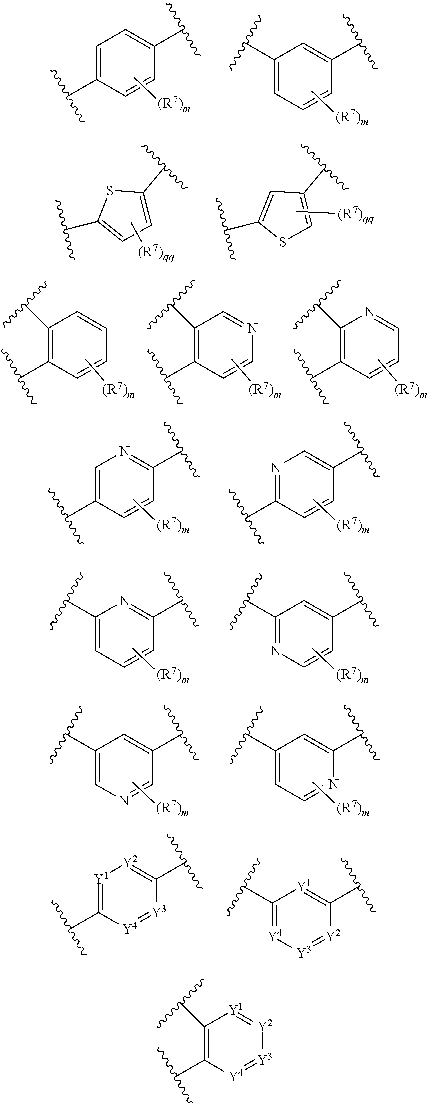

Y¹, Y², Y³ and Y⁴ are independently CR⁷ or N;

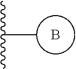

is independently

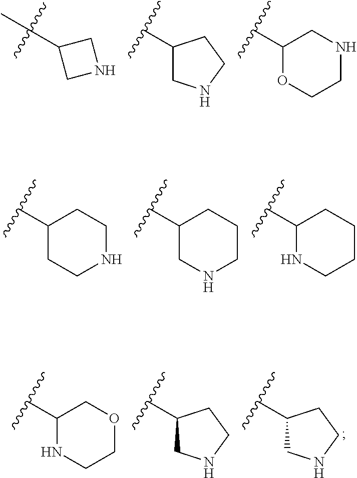

R³ is independently H, -L¹-R^{x}, —(CH₂)ₙOH, —(CH₂)ₙNH₂, —(CH₂)ₙN(R¹⁹)(R²⁰), —(CH₂)ₙC₃₋₆cycloalkyl, —(CH₂)ₙC₆₋₁₀aryl;
L¹ is a chemical bond, —C≡C—, arylene or heteroarylene;
R^{x} is independently H, —(CH₂)ₙ-halogen;
Z is independently H, halogen, C₁₋₄ alkyl, —OC₁₋₄ alkyl, OH, cyclopropyl, —CH₂OH;
Y is independently (C(R⁶)₂)_{g};
g is a integer of 0 to 4;
R⁶ is independently selected from hydrogen, halogen, —C₁₋₆alkyl, —C₁₋₆haloalkyl, —C₃₋₁₀heteroalkyl, —C₃₋₆cycloalkyl, —OC₁₋₆alkyl, —OC₁₋₆haloalkyl, —OC₃₋₆cycloalkyl; or two R⁶ can be connected to form a 3 to 10 membered carbocyclic ring or heterocyclic ring in which the heteroatom is O, S or NR^{d}.

R⁷ is independently OH, CN, SF₅, halo, C₁₋₆alkyl, C₃₋₁₀cycloalkyl, C₃₋₁₀heteroalkyl, C₃₋₁₀heterocylcoalkyl, —C₁₋₆haloalkyl, —C₁₋₆alkoxy, N(R¹⁹)₂, —C₃₋₈cycloalkyl, —C₂₋₄alkenyl, C₂₋₄alkynyl, C₂₋₆heterocyclyl, aryl or heteroaryl;
qq is independently integer of 0 to 2;
n is independently an integer of 0 to 6;
m is independently an integer of 0 to 4;
R¹⁹ and R²⁰ are independently selected from hydrogen, C₁₋₆alkyl, C₃₋₁₀heteroalkyl, C₃₋₆cycloalkyl, C₆₋₁₀aryl or a C₁₋₅heteroaryl, C₂₋₆heterocyclyl;
R^{d} is independently hydrogen, acyl, hydroxyl, C₁₋₈hydroxyalkyl, C₁₋₈alkyl, aralkyl, C₁₋₈haloalkyl, C₁₋₈heteroalkyl, C₃₋₁₀cycloalkyl, C₁₋₁₀heterocyclylalkyl, C₁₋₈alkoxy, C(O)NH₂, C(O)NHC₁₋₆alkyl, C(O)NHC₃₋₆cycloalkyl, C(O)N(C₁₋₆alkyl)₂, S(O)₂C₁₋₆alkyl, S(O)₂C₁₋₆cycloalkyl, S(O)₂NH₂, S(O)₂NHC₁₋₆alkyl, S(O)₂NHC₃₋₆cycloalkyl, NHC(O)NH₂, NHC(O)NHC₁₋₆alkyl, NHC(O)NHC₁₋₆cycloalkyl, NHC(O)OC₁₋₆alkyl, C(O)—C₁₋₆cycloalkyl, C(O)C₁₋₆alkylamino, C₁₋₆heteroalkyl, P(O)(C₁₋₆alkyl)₂, heterocyclyl, heterocyclylalkyl, N(C₁₋₈alkyl)₂ or C₁₋₈alkyl-N(C₁₋₈alkyl)₂; v is an integer of 1 to 6;

In one aspect, the invention provided a compound of Formula XXXXI, or a pharmaceutically acceptable salt thereof:

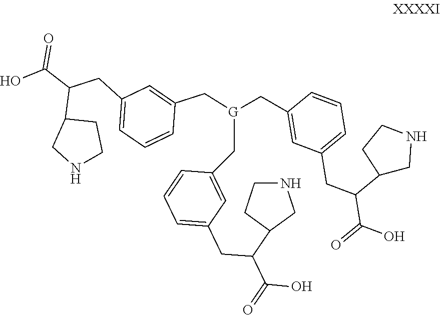

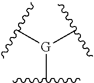

is selected from the following moieties:

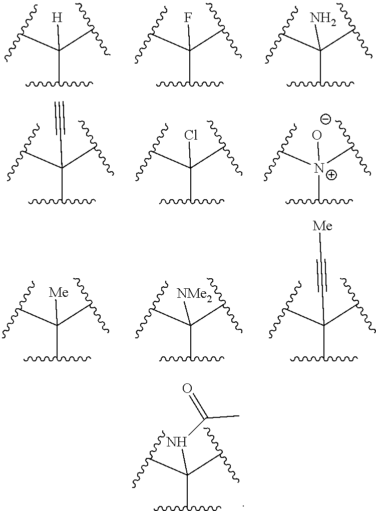

In one aspect, the invention provided a compound of Formula XXXXII, or a pharmaceutically acceptable salt thereof:

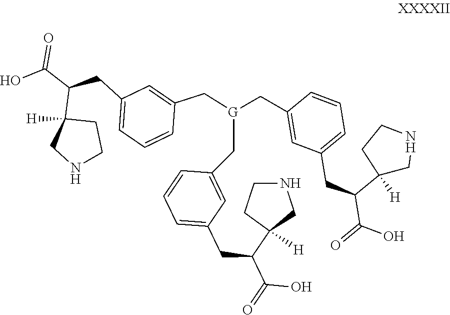

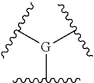

is selected from the following moieties:

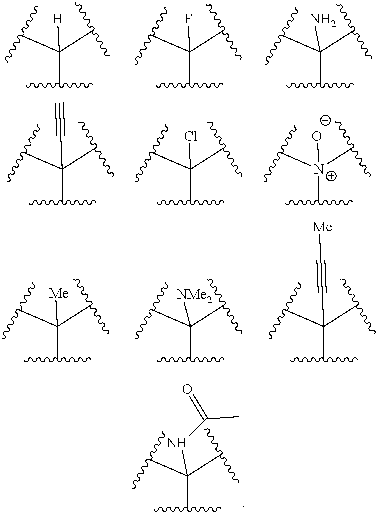

In another aspect, the invention provided a compound of Formula XXXXIII, or a pharmaceutically acceptable salt thereof:

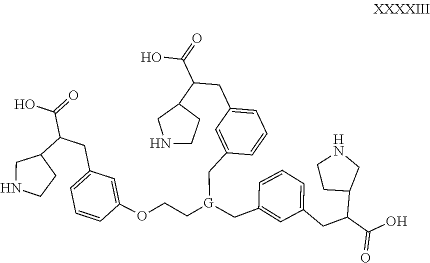

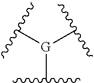

is selected from the following moieties:

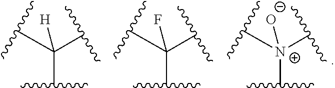

In another aspect, the invention provided a compound of Formula XXXXIV, or a pharmaceutically acceptable salt thereof:

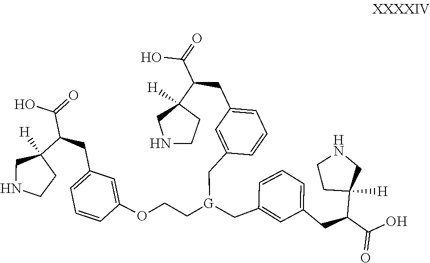

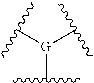

is selected from the following moieties:

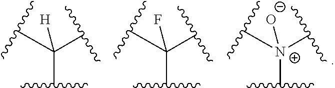

In another aspect, the invention provided a compound selected from the following chemical structures, or a pharmaceutically acceptable salt thereof:

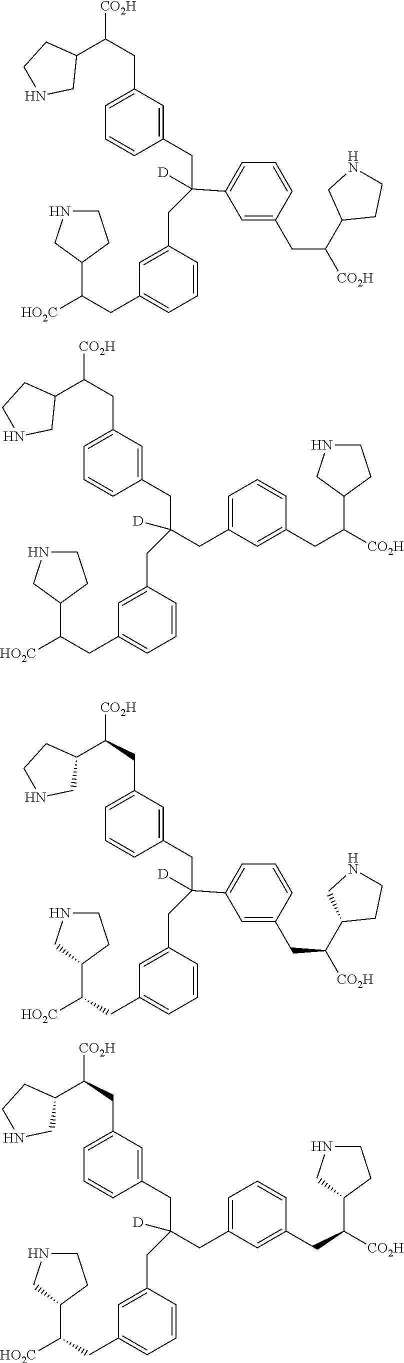

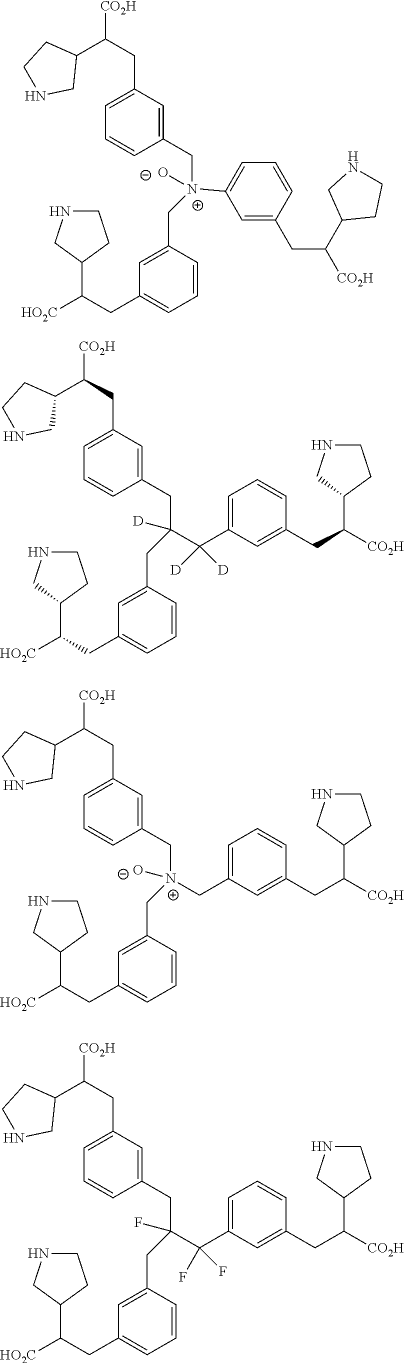

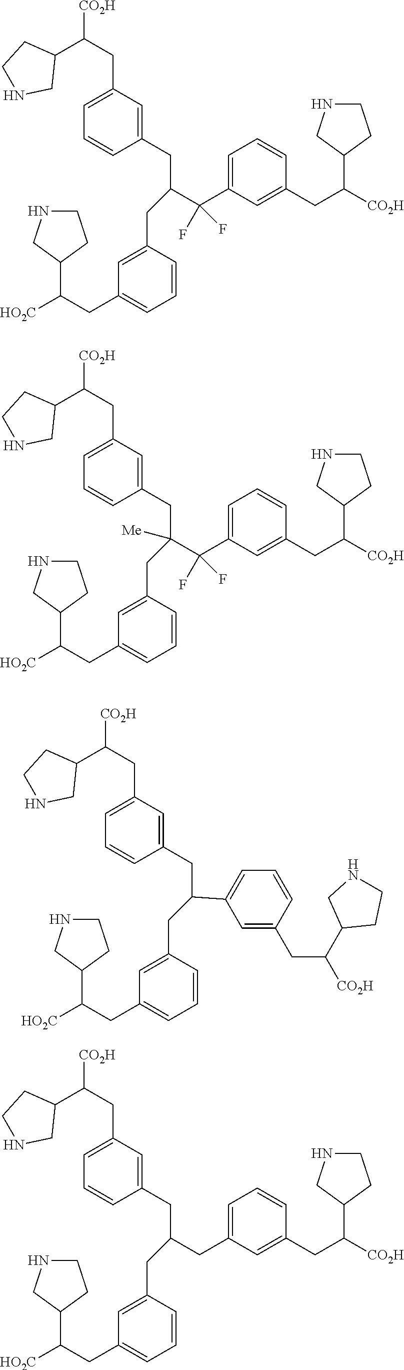

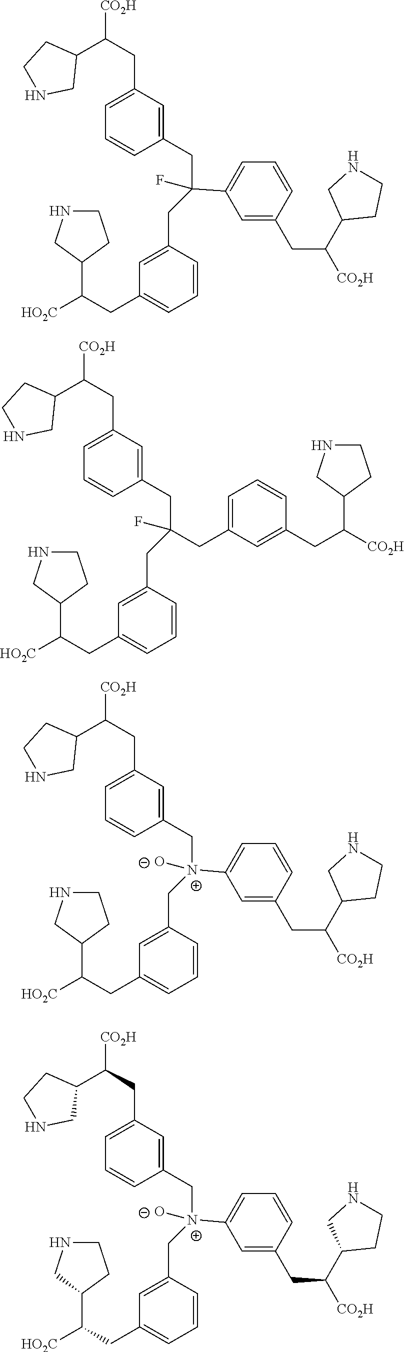

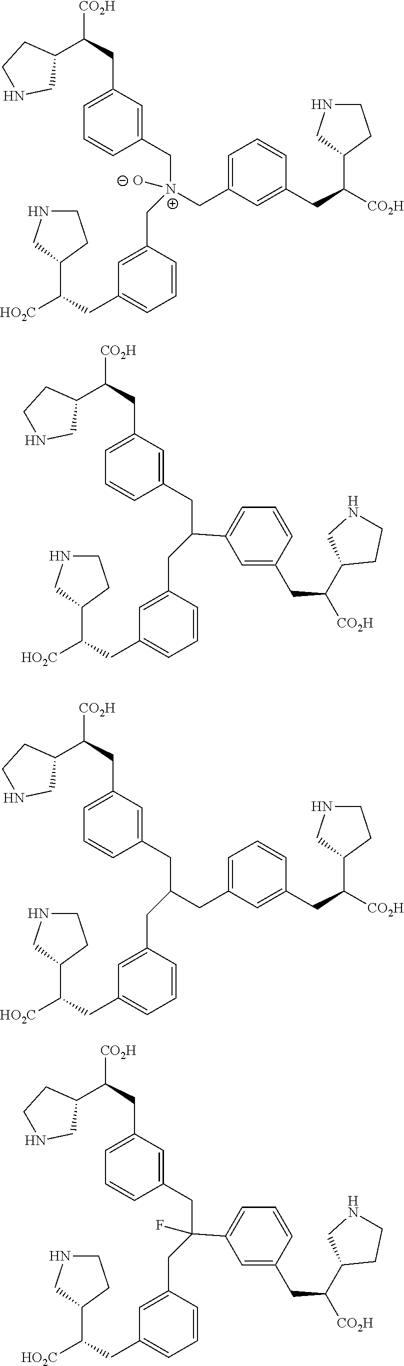

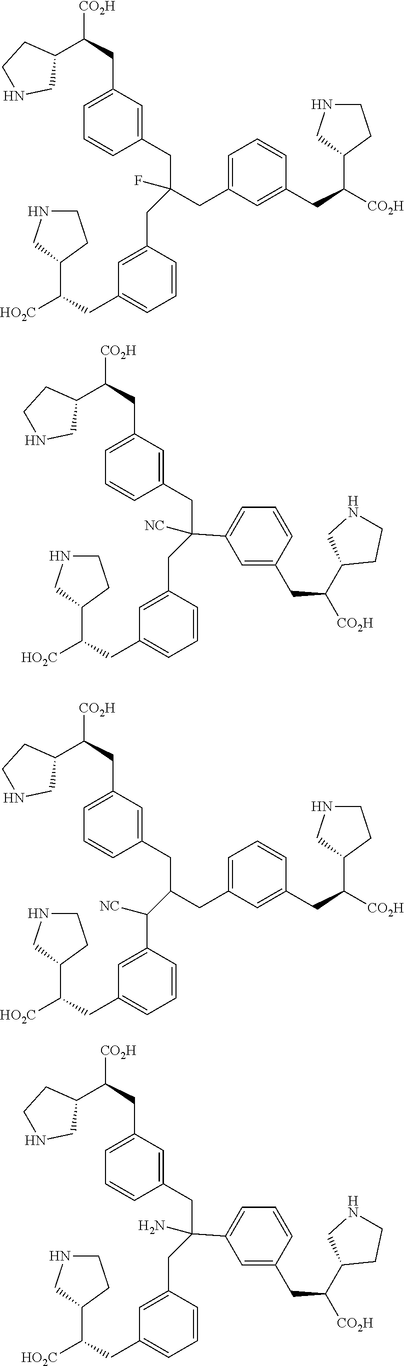

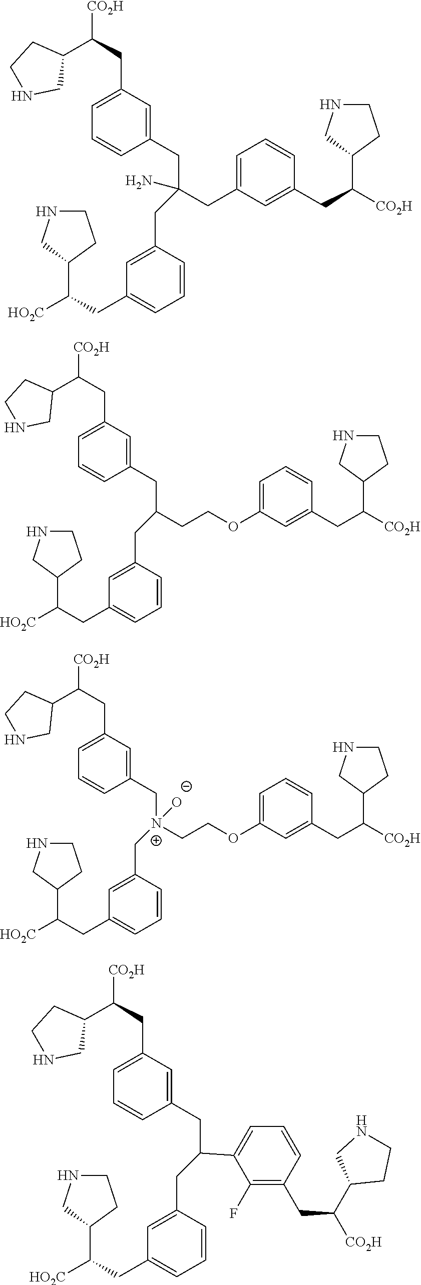

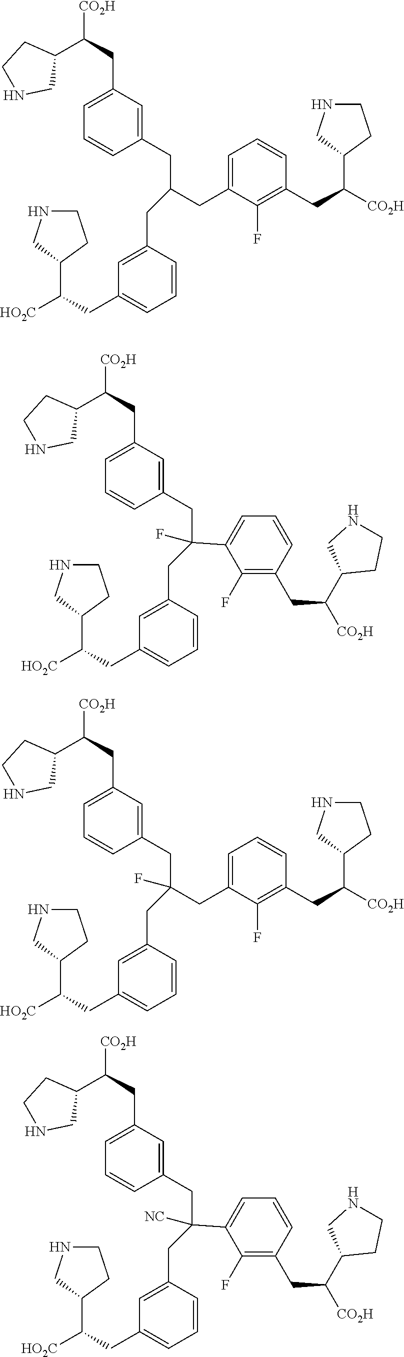

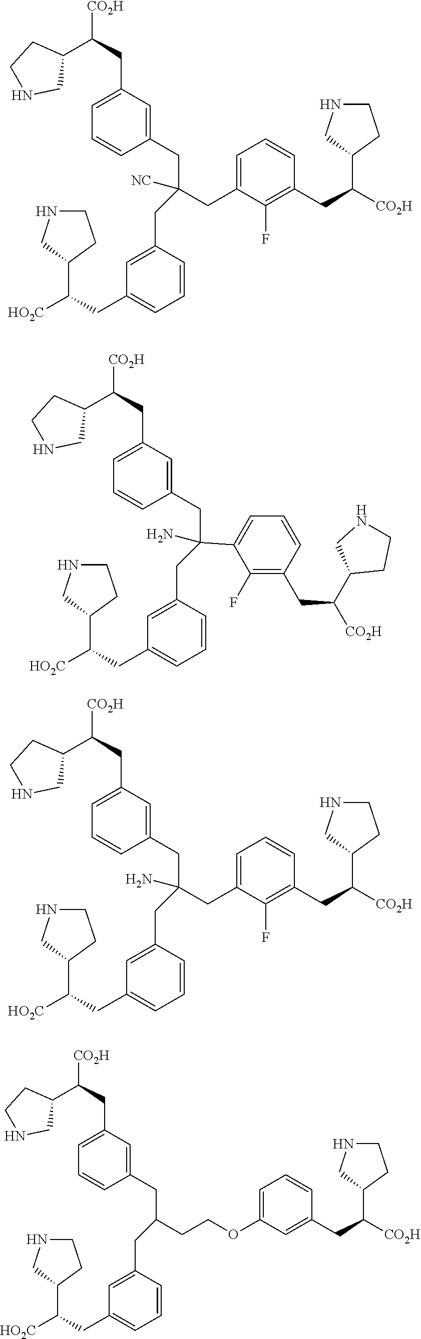

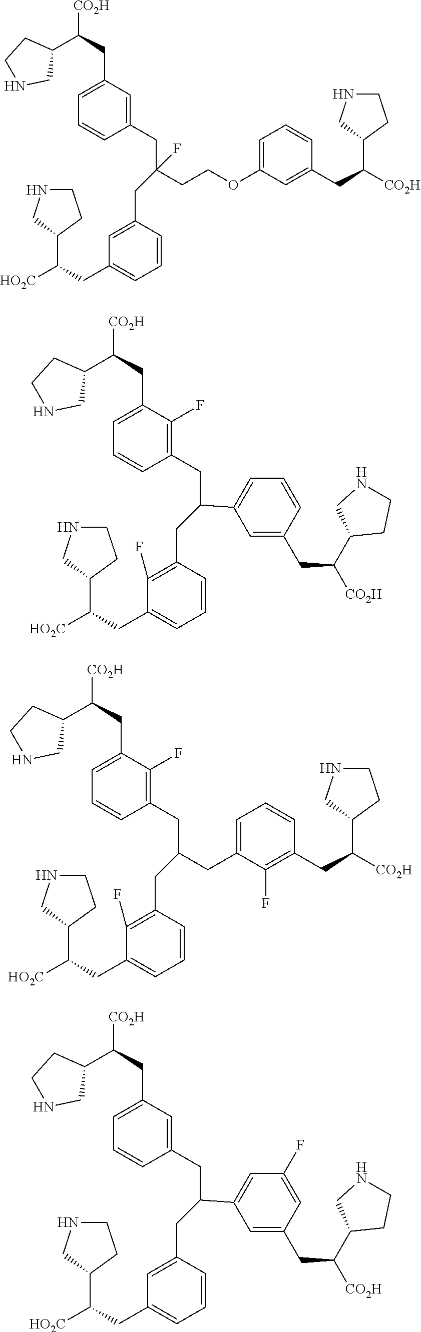

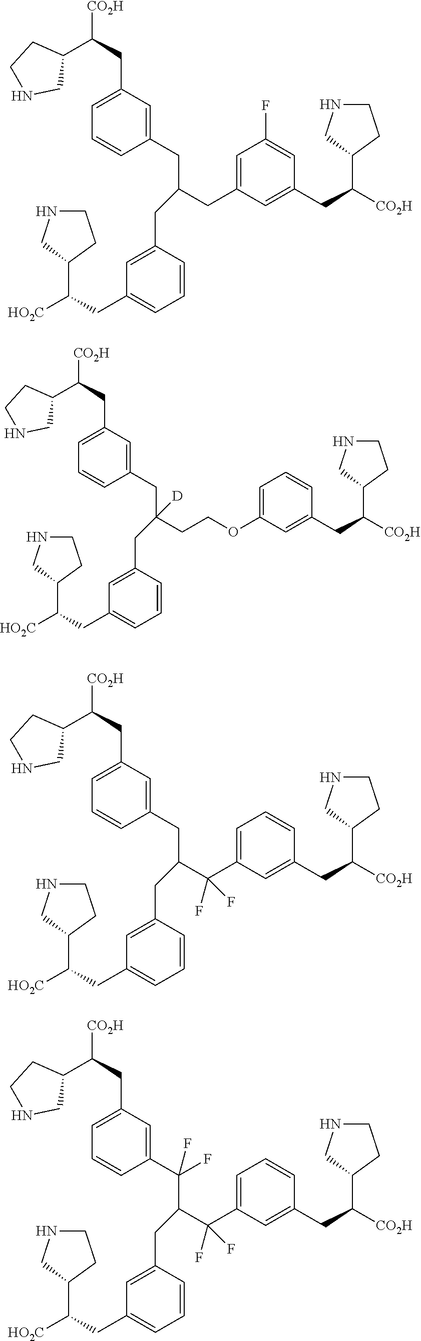

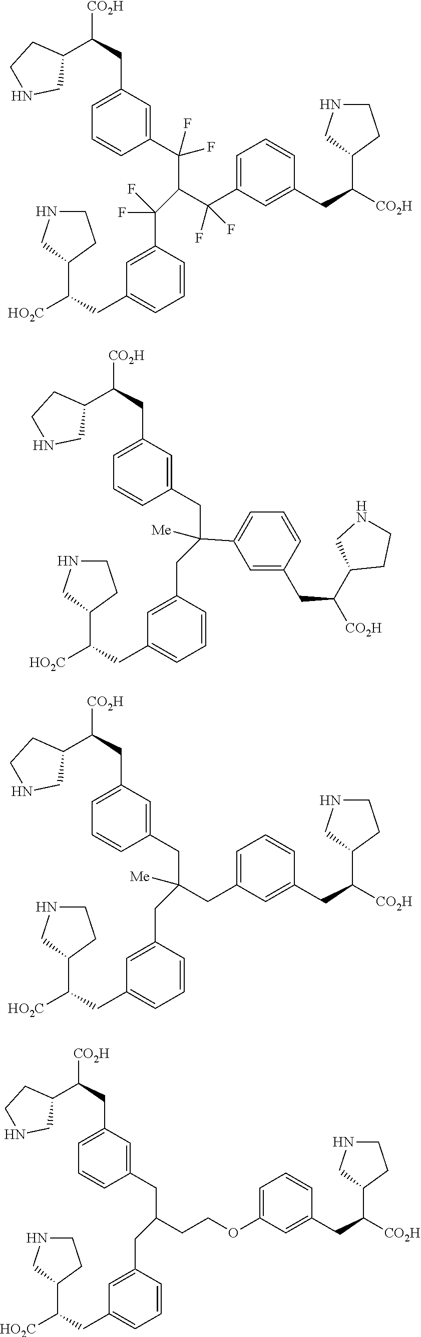

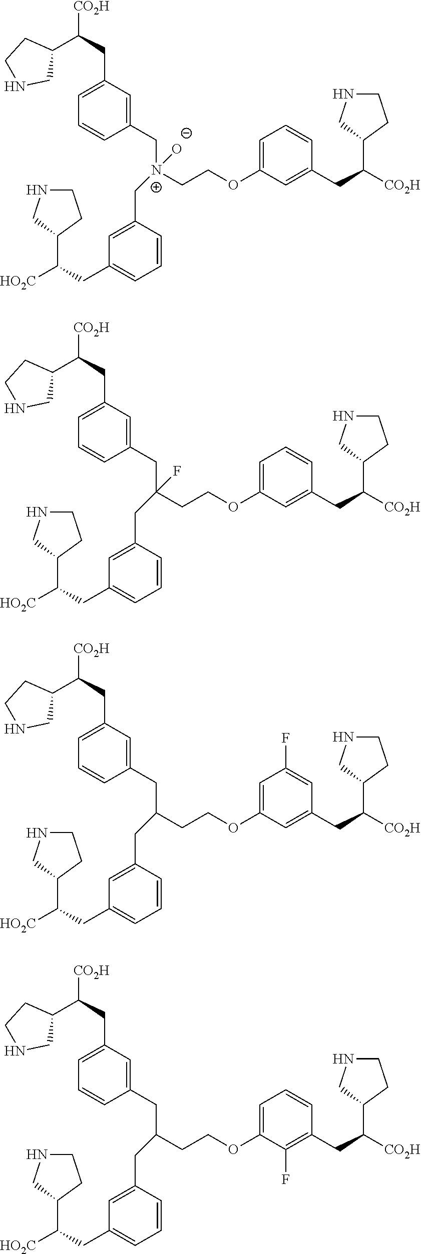 [SMILES: O=C(O)[C@@H](Cc1cccc(C[N+]([O-])(CCOc2cccc(C[C@H](C(=O)O)[C@H]3CCNC3)c2)Cc2cccc(C[C@H](C(=O)O)[C@H]3CCNC3)c2)c1)[C@H]1CCNC1 ; O=C(O)[C@@H](Cc1cccc(CC(F)(CCOc2cccc(C[C@H](C(=O)O)[C@H]3CCNC3)c2)Cc2cccc(C[C@H](C(=O)O)[C@H]3CCNC3)c2)c1)[C@H]1CCNC1 ; O=C(O)[C@@H](Cc1cccc(CC(CCOc2cc(F)cc(C[C@H](C(=O)O)[C@H]3CCNC3)c2)Cc2cccc(C[C@H](C(=O)O)[C@H]3CCNC3)c2)c1)[C@H]1CCNC1 ; O=C(O)[C@@H](Cc1cccc(CC(CCOc2cccc(C[C@H](C(=O)O)[C@H]3CCNC3)c2F)Cc2cccc(C[C@H](C(=O)O)[C@H]3CCNC3)c2)c1)[C@H]1CCNC1]

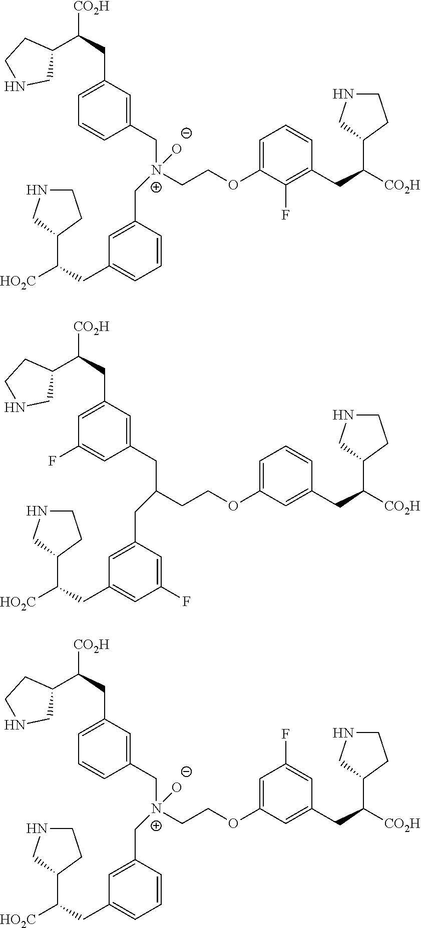 [SMILES: O=C(O)[C@@H](Cc1cccc(C[N+]([O-])(CCOc2cccc(C[C@H](C(=O)O)[C@H]3CCNC3)c2F)Cc2cccc(C[C@H](C(=O)O)[C@H]3CCNC3)c2)c1)[C@H]1CCNC1 ; O=C(O)[C@@H](Cc1cc(F)cc(CC(CCOc2cccc(C[C@H](C(=O)O)[C@H]3CCNC3)c2)Cc2cc(F)cc(C[C@H](C(=O)O)[C@H]3CCNC3)c2)c1)[C@H]1CCNC1 ; O=C(O)[C@@H](Cc1cccc(C[N+]([O-])(CCOc2cc(F)cc(C[C@H](C(=O)O)[C@H]3CCNC3)c2)Cc2cccc(C[C@H](C(=O)O)[C@H]3CCNC3)c2)c1)[C@H]1CCNC1]

In another aspect, the invention provided a pharmaceutical composition comprising a pharmaceutically acceptable carrier and a compound disclosed herein or a pharmaceutically acceptable carrier.

In another aspect the invention provided a method for treating cardiovascular disease mediated by Lp(a). The method comprises administering a therapeutically effective amount of a compound disclosed herein with an option in combination with another therapeutic agent to a subject. In another aspect, the invention features a method of treating any of the following conditions by administering a therapeutically effective amount of a compound disclosed herein with an option in combination with another therapeutic agent to a subject: angina, myocardial infarction, and stroke.

### DEFINITIONS

The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₁₋₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1-and 3-propynyl, 3-butynyl, and the higher homologs and isomers. Alkyl groups which are limited to hydrocarbon groups are termed “homoalkyl”. The said alkyl is optionally substituted with one or more halogen atom(s).

The term “Halogenated alkyl” means alkyl as defined above wherein one or more hydrogen atoms have been replaced by halogen atoms.

The term “Alkylene” by itself or as part of another substituent means a divalent radical derived from an alkyl, as exemplified, but not limited, by —CH₂CH₂CH₂CH₂—, —CH₂CH═CHCH₂—, —CH₂ C≡CCH₂—, —CH₂CH₂CH(CH₂CH₂CH₃)CH₂—. Typically, an alkyl (or alkylene) group has from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The said alkylene is optionally substituted with one or more halogen atom(s).

The term “Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like. The said alkynyl is optionally substituted with one or more halogen atom(s).

The term “Cycloalkyl” means mono- or bicyclic saturated carbocyclic rings, each of which has from 3 to 10 carbon atoms. A “fused analog” of cycloalkyl means a monocyclic ring fused to an aryl or heteroaryl group in which the point of attachment is on the non-aromatic portion. Examples of cycloalkyl and fused analogs thereof include cyclopropyl, cyclobutyl, cyclopentyl, cyclohexyl, cycloheptyl, tetrahydronaphthyl, decahydronaphthyl, indanyl, and the like. The said cycloalkyl is optionally substituted with one or more halogen atom(s).

The term “Alkoxy” means alkoxy groups of a straight or branched having the indicated number of carbon atoms. C₁₋₆alkoxy, for example, includes methoxy, ethoxy, propoxy, isopropoxy, and the like.

The term “Heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S —CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both-C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like, where at least one of R′ or R″ is an alkyl.

The term “cycloalkoxy” means cycloalkyl as defined above bonded to an oxygen atom, such as cyclopropyloxy.

The term “Halogenated alkoxy” means alkoxy as defined above wherein one or more hydrogen atoms have been replaced by halogen atoms.

The term “aryl” means mono- or bicyclic aromatic rings containing only carbon atoms. A “fused analog” of aryl means an aryl group fused to a monocyclic cycloalkyl or monocyclic heterocyclyl group in which the point of attachment is on the aromatic portion. Examples of aryl and fused analogs thereof include phenyl, naphthyl, indanyl, indenyl, tetrahydronaphthyl, 2,3-dihydrobenzofuranyl, dihydrobenzopyranyl, 1,4-benzodioxanyl, and the like.

The term “heteroaryl” means a mono- or bicyclic aromatic ring containing at least one (such 1, 2 or 3) heteroatom selected from N, O and S, with each ring containing 5 to 6 atoms. A “fused analog” of heteroaryl means a heteroaryl group fused to a monocyclic cycloalkyl or monocyclic heterocyclyl group in which the point of attachment is on the aromatic portion. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

The said alkyl groups, aryl groups and said heteroaryl groups referred to in the definitions are unsubstituted or are substituted by at least one substituent selected from the group consisting of substituents.

The said substituents are selected from the group consisting of halogen atoms, hydroxyl group, alkyl groups having from 1 to 4 carbon atoms, alkoxy groups having from 1 to 4 carbon atoms, haloalkyl groups having from 1 to 4 carbon atoms, haloalkoxy groups having from 1 to 4 carbon atoms, cyano groups, alkynyl groups having from 2 to 6 carbon atoms, alkanoyl groups having from 1 to 5 carbon atoms, cycloalkyl groups having from 3 to 7 ring atoms, heteroaryl groups, aryl groups, aralkoxy groups having from 7 to 10 carbon atoms, arylcarbonyl groups, two adjacent-x groups are optionally joined together to form an alkylene or an alkenylene chain having 3 or 4 carbon atoms, aminocarbonyl groups, alkenyl groups having from 2 to 5 carbon atoms, alkylthio groups having from 1 to 4 carbon atoms, aminosulfinyl groups, aminosulfonyl groups, hydroxy groups, —SF₅, hydroxyalkyl groups having from 1 to 4 carbon atoms, nitro groups, amino groups, carboxy groups, alkoxycarbonyl groups having from 2 to 5 carbon atoms, alkoxyalkyl groups having from 1 to 4 carbon atoms, alkylsulfonyl groups having from 1 to 4 carbon atoms, alkanoylamino groups having from 1 to 4 carbon atoms, alkanoyl(alkyl)amino groups having from 1 to 6 carbon atoms, alkanoylaminoalkyl groups having from 1 to 6 carbon atoms in both the alkanoyl and alkyl part, alkanoyl(alkyl)aminoalkyl groups having from 1 to 6 carbon atoms in both the alkanoyl and each alkyl part, alkylsulfonylamino groups having from 1 to 4 carbon atoms, mono- or di-alkylaminocarbonyl groups having from 1 to 6 carbon atoms, mono- or di-alkylaminosulfinyl groups having from 1 to 6 carbon atoms, mono- or di alkylaminosulfonyl groups having from 1 to 6 carbon atoms, aminoalkyl groups having from 1 to 4 carbon atoms, mono- or di-alkylamino groups having from 1 to 6 carbon atoms, mono- or di-alkylaminoalkyl groups having from 1 to 6 carbon atoms in each alkyl part, aralkyl groups having from 7 to 10 carbon atoms, heteroarylalkyl groups having from 1 to 4 carbon atoms in the alkyl part, heteroarylalkoxy groups having from 1 to 4 carbon atoms in the alkoxy part and alkylsulfonylamino groups having from 1 to 4 carbon atoms;

The term “heterocyclyl” means mono- or bicyclic saturated rings containing at least one heteroatom selected from N, S and O, each of said ring having from 3 to 10 atoms in which the point of attachment may be carbon or nitrogen. A “fused analog” of heterocyclyl means a monocyclic heterocycle fused to an aryl or heteroaryl group in which the point of attachment is on the non-aromatic portion. Examples of “heterocyclyl” and fused analogs thereof include pyrrolidinyl, piperidinyl, piperazinyl, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

The terms “halo” or “halogen,” by themselves or as part of another substituent, mean, unless otherwise stated, a fluorine, chlorine, bromine, or iodine atom. Additionally, terms such as “haloalkyl,” or “halogenated alkyl” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrugs may also have improved solubility in pharmaceutical compositions over the parent drug. An example, without limitation, of a prodrug would be a compound of any of Formula I to VI, which is administered as an ester (the “prodrug”) to facilitate transmittal across a cell membrane where water solubility is detrimental to mobility, but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

Optical Isomers-Diastereomers-Atropisomers-Geometric Isomers-Tautomers-Zwitteruinic forms:

Compounds of all formula described in the present invention may contain one or more asymmetric centers/hindered rotation about a single bond, and may thus occur as racemates and racemic mixtures, single enantiomers, single atropisomers, diastereomeric mixtures and individual diastereomers. The present invention is meant to comprehend all such isomeric forms of the compounds.

Some of the compounds described herein contain olefinic double bonds, and unless specified otherwise, are meant to include both E and Z geometric isomers.

Some of the compounds described here may contain one or more than one cyclic ring systems and may thus exist in cis- and trans-isomers. The present invention is meant to include all such cis- and trans-isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of the invention.

Compounds described here may be separated into diastereoisomeric pairs of enantiomers by, for example, HPLC or fractional crystallization from a suitable solvent, for example MeOH or EtOAc or a mixture thereof. The pair of enantiomers thus obtained may be separated into individual stereoisomers by conventional means, for example by the use of an optically active amine or acid as a resolving agent or on a chiral HPLC column.

The zwitterionic interchanging form can derive from one or more than one pair of zwitterion forming groups:

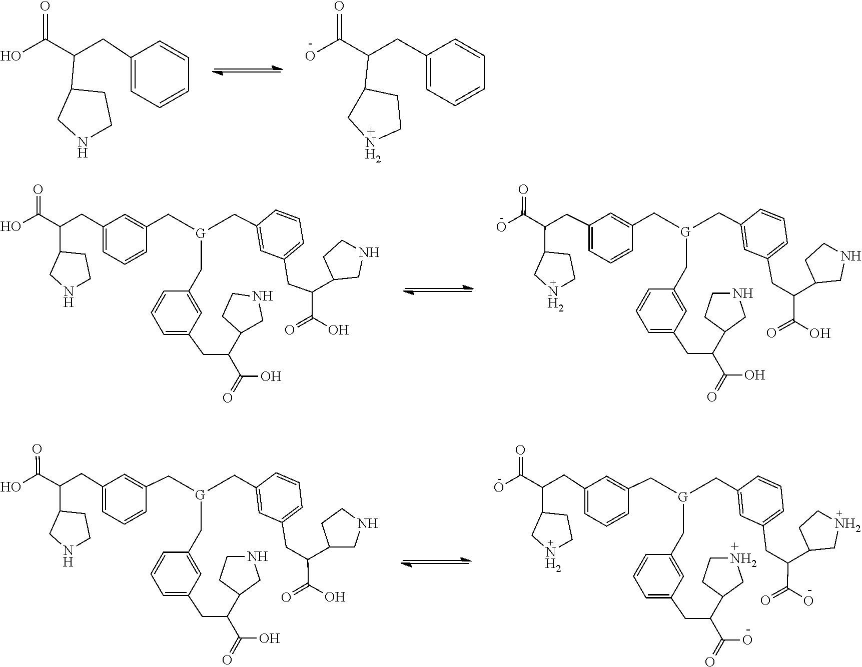

Alternatively, any enantiomer of a compound described here may be obtained by stereospecific synthesis using optically pure starting materials or reagents of known configuration.

### Stable Isotope-Labeled Analogs

One or more than one of the protons in compounds described here can be replaced with deuterium atom(s), thus providing deuterated analogs that may have improved pharmacokinetics properties or pharmacological activities.

### Salts and Formulations

The compounds described herein can be useful as the free base or as a salt.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydramine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is alkaline, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds described here are meant to also include the pharmaceutically acceptable salts.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients are mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methylcellulose, hydroxypropylmethy-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Compounds described here may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Dosage levels of the order of from about 0.01 mg to about 140 mg/kg of body weight per day or about 0.5 mg to about 7 g per patient per day are useful in the treatment of the above-indicated conditions.

When the active ingredient is combined with the carrier material to form a single dosage form, the amount will vary depending on the treatment target and specific administration method. For instance, an oral formulation may contain 0.5 mg to 5 g of the active ingredient, which is blended with an appropriate amount of carrier material (accounting for about 5% to 95% of the total formulation). The dosage unit generally contains about 1 to 1000 mg of the active ingredient (typically 25 mg, 50 mg, 100 mg, 200 mg, 300 mg, 400 mg, 500 mg, 600 mg, 800 mg, or 1000 mg).

However, the specific dosage for any particular patient depends on a variety of factors, including age, weight, overall health status, sex, diet, timing of administration, route of administration, excretion rate, drug combinations, and the severity of the specific disease being treated.

### SYNTHESIS

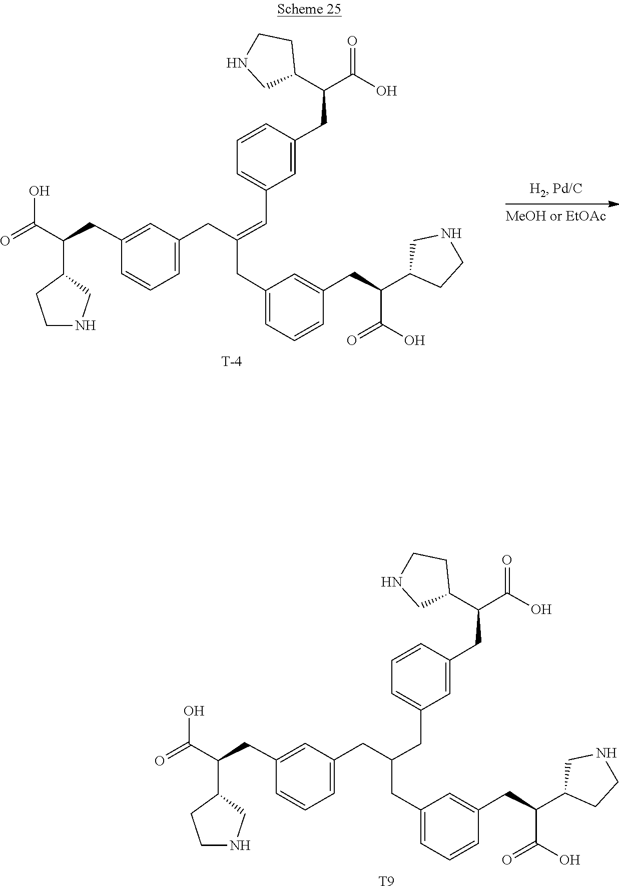

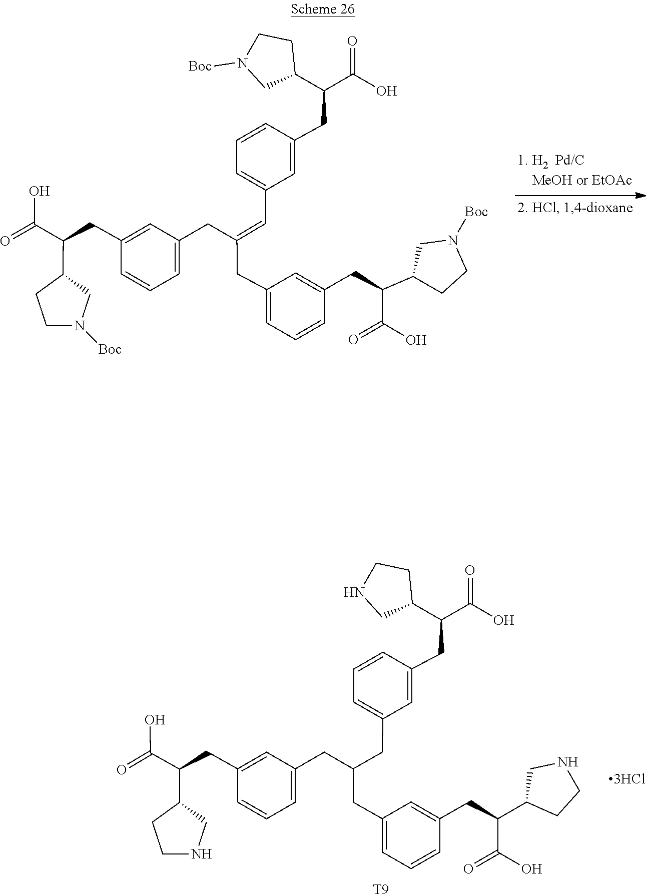

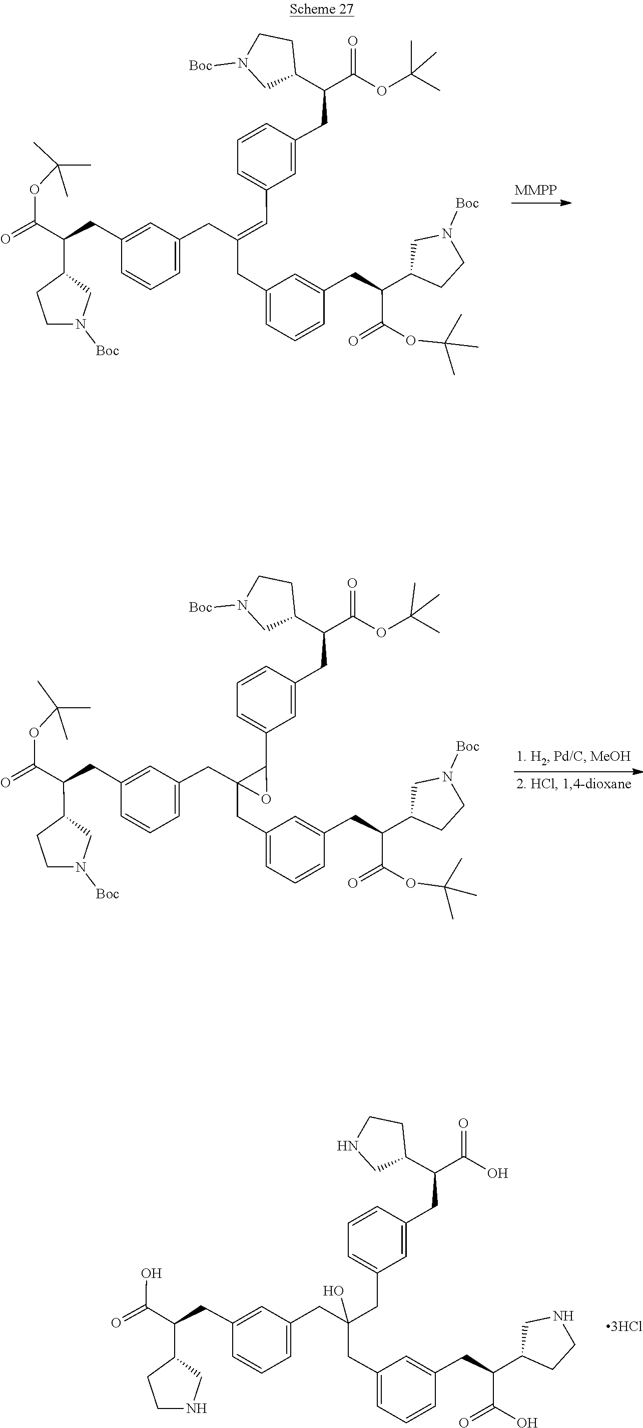

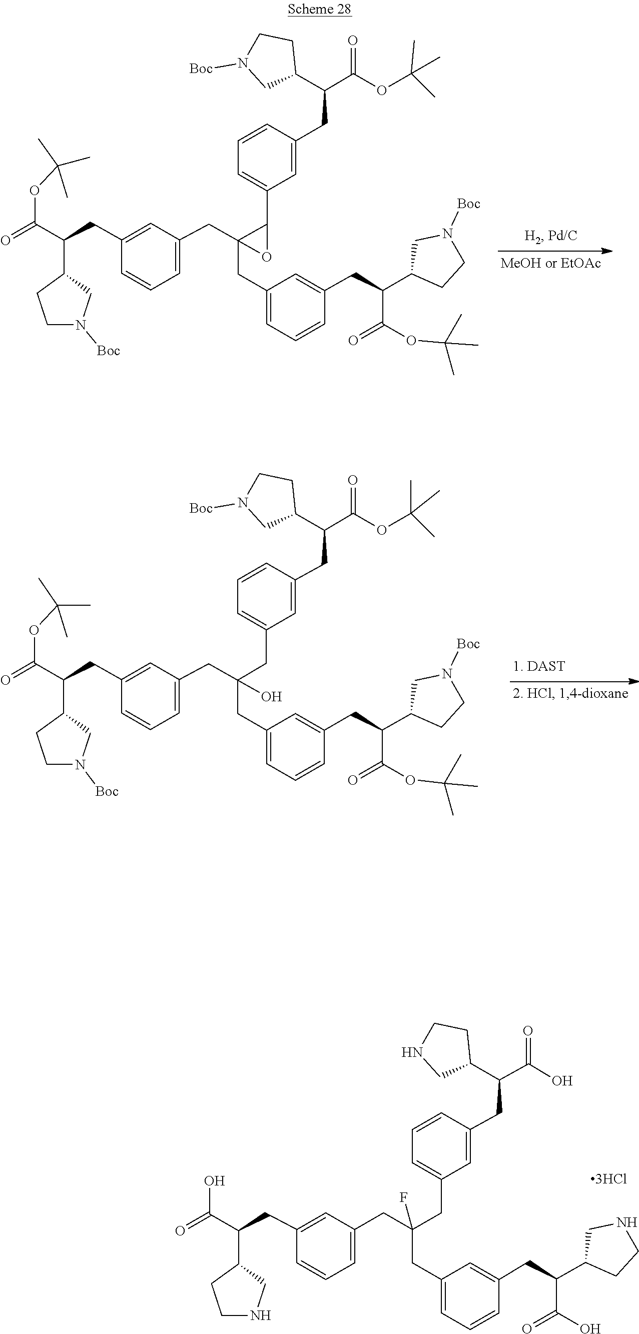

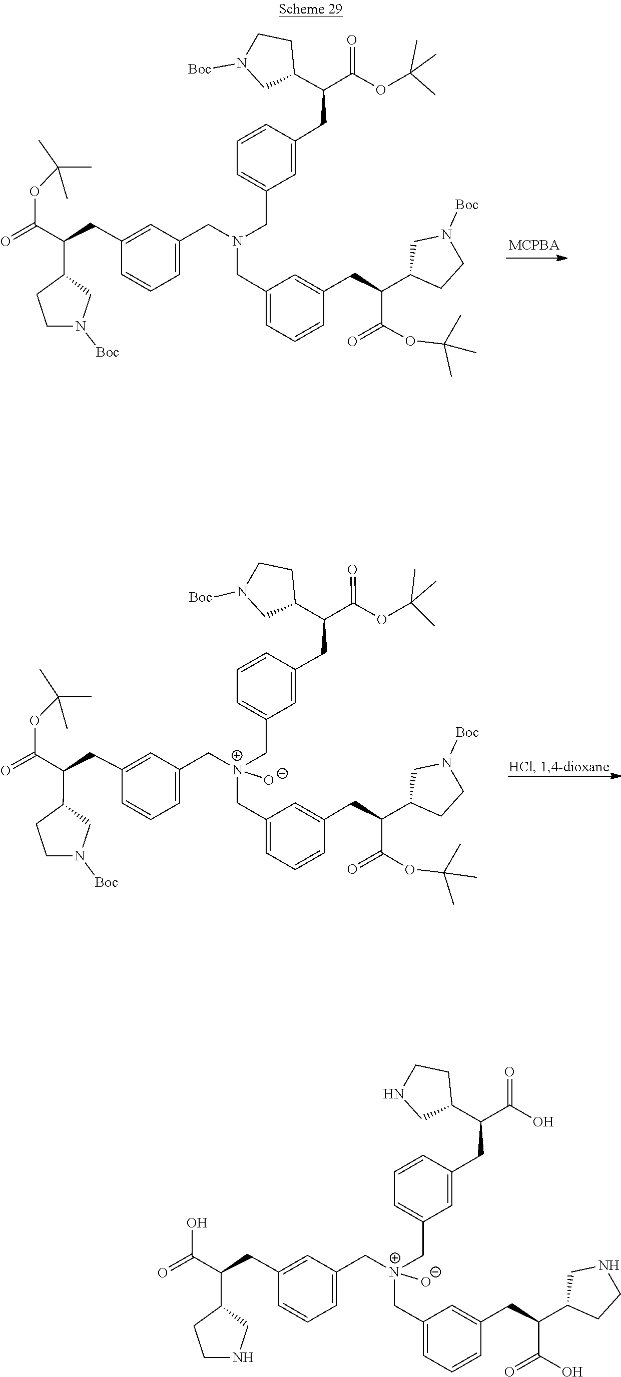

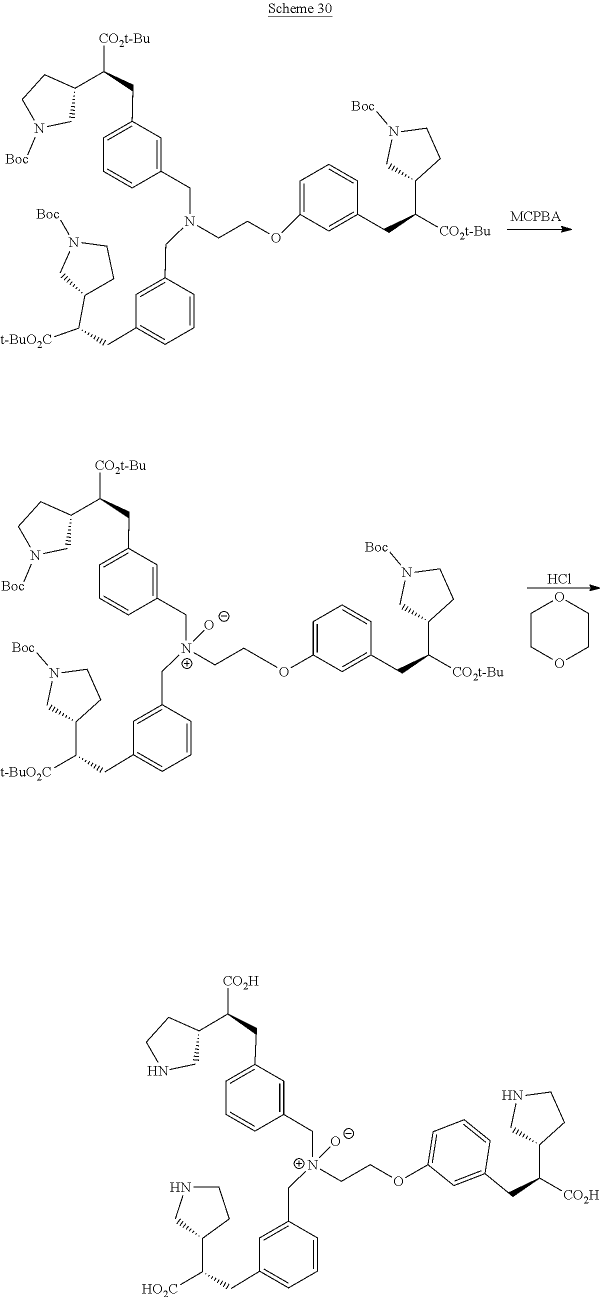

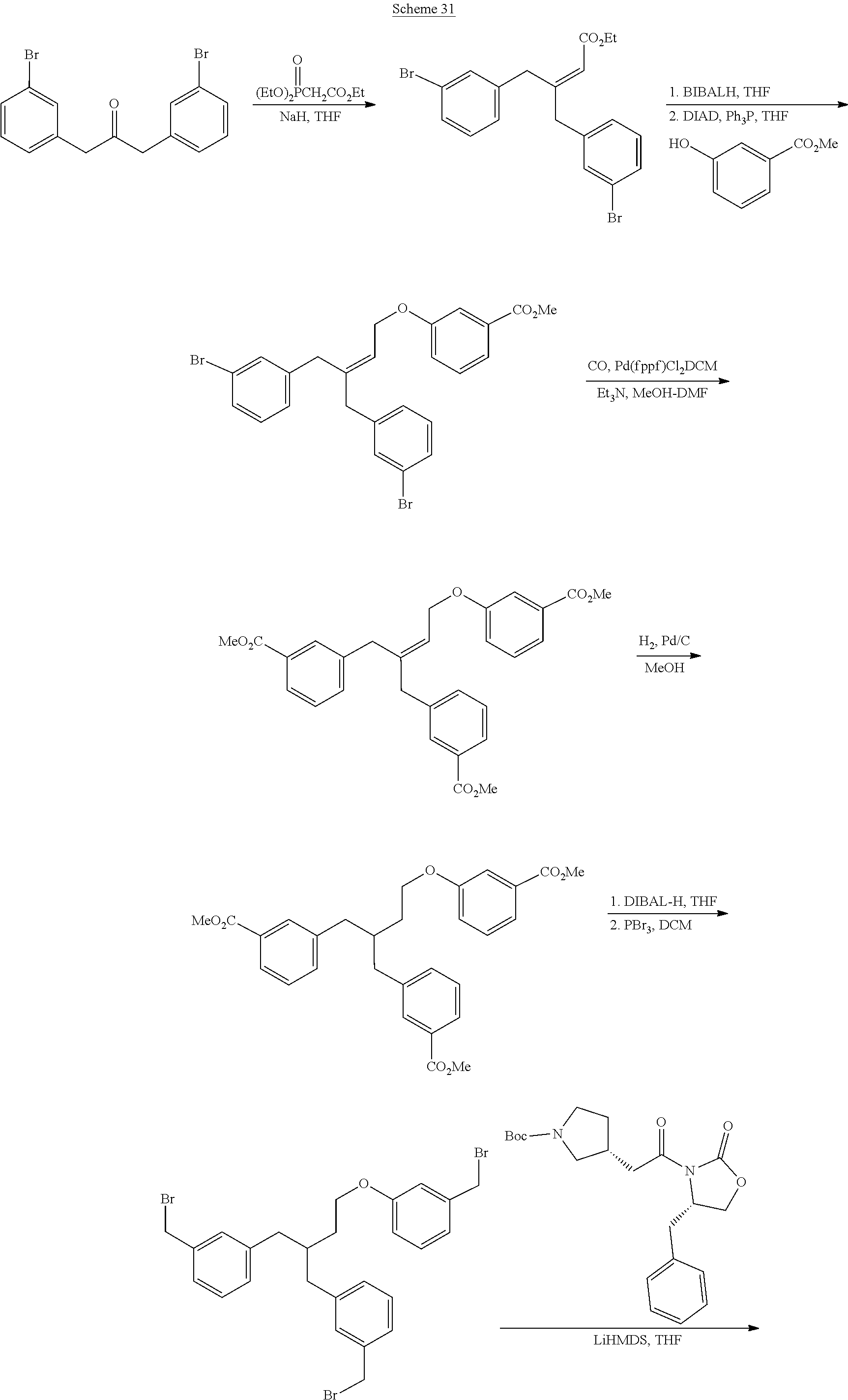

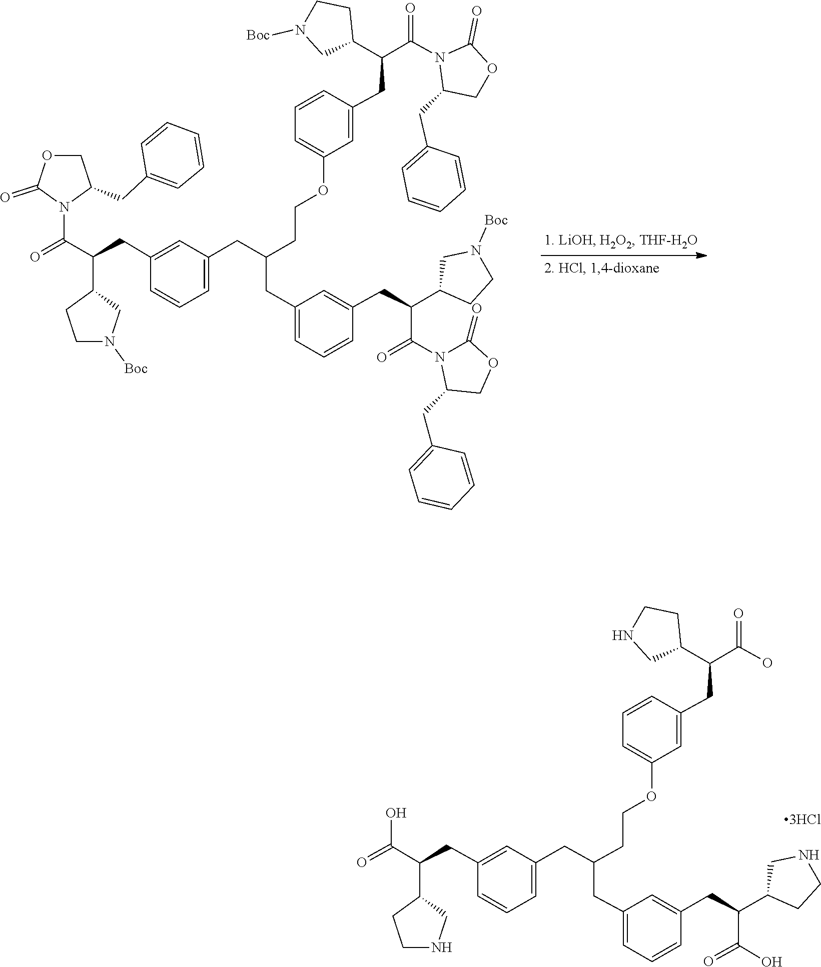

### ABBREVIATIONS

The following abbreviations have the meanings indicated. EA means ethyl acetate; CIP means 2-chloro-1,3-dimethylimidazolidium hexafluorophosphate; DAST means diethylaminosulfur trifluoride; DBU means 1,8-diazabicyclo[5.4.0]undec-7-ene; DCM means dichloromethane; DIAD means diisopropyl azodicarboxylate; DIBAL or DIBAL-H means diisobutylaluminum hydride; DCM means dichloromethane; DDQ means 1,2-dichloro-4,5-dicyanobenzoquinone; DEAD means diethyl azodicarboxylate; DIAD means diisopropyl azodicarboxylate; DIEA means diisopropylethylamine; DMAP means N,N-dimethylaminopyridine; DME means 1,2-dimethoxyethane; DMF means N,N-dimethylformamide; dmpe means 1,2-bis(dimethylphosphino)ethane; DMA means N,N-Dimethylacetamide, DMSO means dimethylsulfoxide; dppb means 1,4-bis(diphenylphosphino)butane; dppe means 1,2-bis(diphenylphosphino)ethane; dppf means 1,1′-bis(diphenylphosphino)ferrocene; dppm means 1,1′-bis(diphenylphosphino)methane; EDCI means 1-(3-dimethylaminopropyl)-3-ethylcarbodiimide; FA means formic acid; HATU means 2-(7-Aza-1H-benzotriazole-1-yl)-1,1,3,3-tetramethyluronium hexafluorophosphate; HMPA means hexamethyl phosphorarnide; IPA means isopropyl alcohol; LDA means lithium diisopropylamide; LHMDS means lithium bis(hexamethyldisilylamide); LAH means lithium aluminum hydride; MCPBA means 3-chloroperbenzoic acid; MMPP means magnesium monoperoxyphthalate; MTBE means methyl tert-butyl ether; NBS means N-Bromosuccinimide; NCS means N-chlorosuccinimide; NMI means 1-methylimidazole; NMM means N-methylmorpholine; NMP means N-methylpyrrolidine; PE means petroleum ether; PyBOP 26yridazine26leriazol-1-yloxytripyrrolidinophosphonium hexafluorophosphate; SGCC means silica gel column chromatography; TBAF means (n-Butyl)₄NF; TDA means tris(2-(2-methoxyethoxy)ethyl)amine; TEA means triethylamine; TFA means trifluoroacetic acid; THF means tetrahydrofuran; PPh3 means triphenylphosphine; rt or r.t. means room temperature; sat. means saturated; T3P means propylphosphonic anhydride; TCFH means N,N,N′,N′-tetramethylchloroformaidinium hexafluorophosphate; * in structures means undetermined R or S chiral center; Pd₂(dba)₃ means tris(dibenzylideneacetone)dipalladium; X-PHOS means 2-(dicyclohexylphosphino)-2′,4′,6′-tri-i-propyl-1,1′-biphenyl.

### Prep-HPLC Conditions:

Column: Waters XTerra® Prep MS C18, 19×250 mm.
Mobile phase: A water (0.1% FA or 0.1% TFA); B MeCN. Flow rate: 15 mL/min.
   LCMS conditions:
Column: Xtimate C18, 3 mm, 4.6×50 mm.
Mobile phase: water (0.1% FA) and MeCN;
Gradient: 5% CH₃CN to 95% CH₃CN in 1.5 min, then 95% MeCN for 2.5 min;
Flow rate: 0.5 mL/min.;
MS instrument: Agilent 6120 single-quadrupole mass spectrometer.

### Intermediate M-1

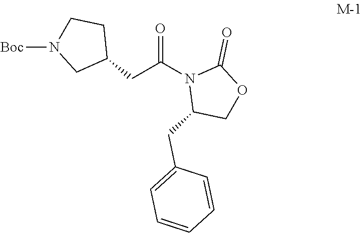 [SMILES: CC(C)(C)OC(=O)N1CC[C@H](CC(=O)N2C(=O)OC[C@@H]2Cc2ccccc2)C1]

To a solution of (R)-2-(1-(tert-butoxycarbonyl)pyrrolidin-3-yl)acetic acid (15 g) in THF (400 mL) was added Et₃N (13.22 g) and pivaloyl chloride (11 g) under ice bath. After stirring at ice bath for 30 min, (S)-4-benzyloxazolidin-2-one (12.75 g) and DMAP (0.8 g) was added. After stirring at rt for for 1 day, saturated NH₄Cl aqueous solution (400 mL) was added and the mixture was extracted with EA (200 mL×3). The EA extract was concentrated and the residue was purified by SGCC eluted with 0 to 80% EA in PE to provide M-1 (19.23 g) as a white solid.

MS ESI: m/z=333, [(M-tert-butyl)+1]⁺.

### Example T-3

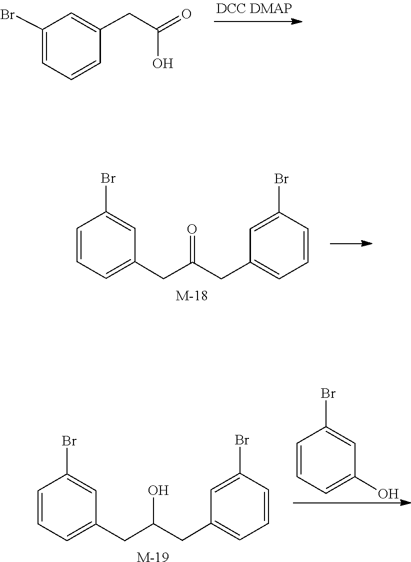

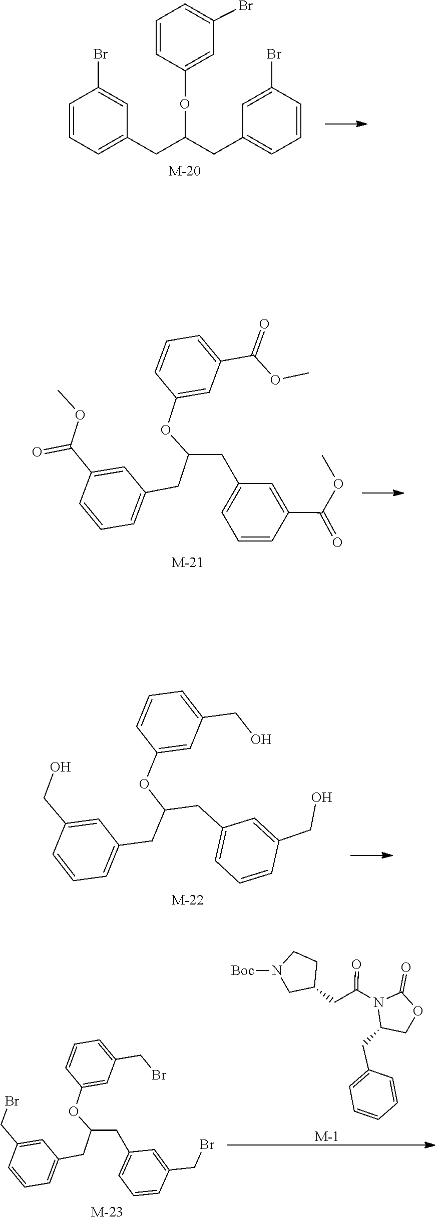

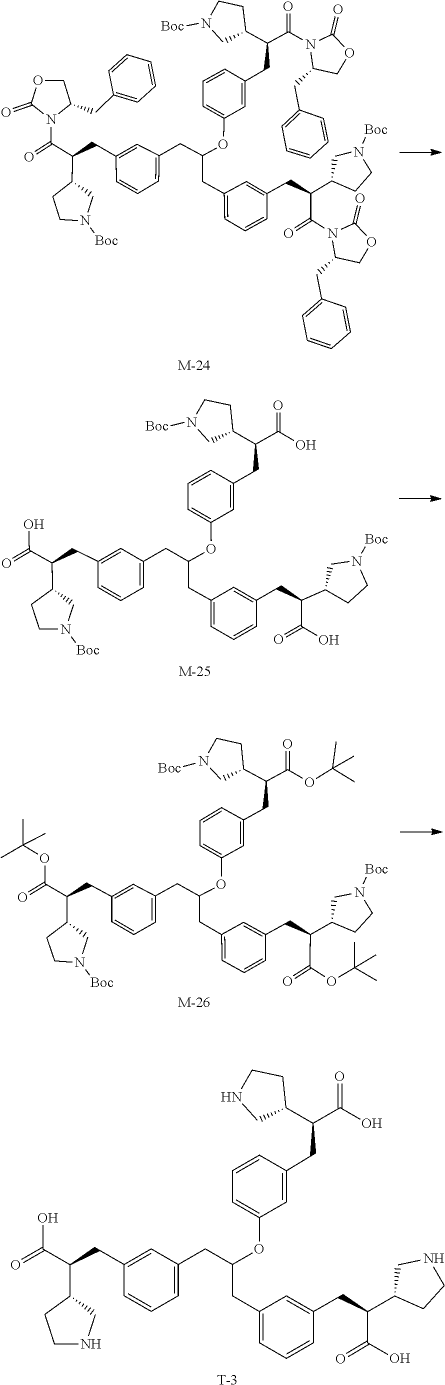

### Step 1: M-18

To a solution of DCC (15.11 g) and DMAP (2.13 g) in DCM (150 mL) was slowly added 2-(3-bromophenyl)acetic acid (15 g) in DCM (150 mL) dropwise. After stirring at rt overnight, the reaction mixture was filtered and the filtrate was concentrated in vacuum. The residue was dissolved in EA (200 mL) and filtered through a pad of silica gel. The filtrate was concentrated in vacuum and the residue was dissolved in EA (20 mL) and PE (100 mL). After stirring for 2 h, the solid was collected by filtration to yield M-18 (7.05 g) as a white solid.

¹H NMR (400 MHz, CDCl₃) δ 7.41 (d, J=6.9 Hz, 2H), 7.30 (s, 2H), 7.24-7.16 (m, 2H), 7.08 (d, J=6.9 Hz, 2H), 3.71 (s, 4H).

### Step 2: M-19

To a solution of M-18 (10 g) in MeOH (100 mL) was added NaBH₄ (1.23 g). After stirring at rt for 2 h, the solvent was removed in vacuum. The residue was treated with saturated NH₄Cl aqueous solution (100 mL) and extracted with EA (50 mL×3). The EA extract was concentrated in vacuum to yield 10.4 g of light yellow oily M-19.

### Step 3: M-20

To a solution of M-19 (5 g) in THF (60 mL) was added 3-bromophenol (4.68 g), DIAD (3.55 g) and PPh₃ (4.61 g). After stirring at rt overnight, the solvent was removed in vacuum. The residue was purified by SGCC eluted with 0 to 10% EA in PE to provide 2.24 g of colorless oily M-20.

¹H NMR (400 MHz, acetone-d₆) δ 7.49 (s, 2H), 7.38 (d, J=6.4 Hz, 2H), 7.30 (d, J=6.0 Hz, 2H), 7.24 (m, 2H), 7.14 (m, 1H), 7.03 (m, 2H), 6.89 (d, J=6.7 Hz, 1H), 5.00 (m, 1H), 3.12-2.94 (m, 4H).

### Step 4: M-21

To a solution of M-20 (1.5 g) in DMF (15 mL) and MeOH (9 mL) was added Pd(dppf)Cl₂ DCM (463 mg), dppf (0.32 g) and NEt₃ (1.73 g). After stirring under CO atmosphere (balloon) at 85 °C for 3 days, saturated NH₄Cl aqueous solution (50 mL) was added and the mixture was extracted with EA (50 mL×3). The EA extract was concentrated and the residue was purified by SGCC eluted with 0 to 30% EA in PE to provide M-21 (1.28 g).

### Step 5: M-22

To a solution of M-21 (1.58 g) in toluene (20 mL) cooled in an ice bath was added DIBAL-H (21.52 mmol). After stirring at rt for 2.5 h, the reaction was quenched with saturated aqueous potassium sodium tartrate solution (50 mL). After stirring at rt for 2 h, the mixture was extracted with EA (100 mL). The EA extract was concentrated in vacuum to yield M-22 which was used in the next step without further purification.

### Step 6: M-23

To a solution of M-22 in DCM (20 mL) was added PBr₃ (1.38 g). After stirring at rt for 2 h, the reaction mixture was quenched with saturated NaCl aqueous solution (50 mL) and extracted with DCM (20 mL×3). The DCM extract was concentrated and the residue was purified by SGCC eluted with 0 to 10% EA in PE to provide M-23 (1.22 g).

¹H NMR (400 MHz, CDCl₃) δ 7.30-7.21 (m, 6H), 7.17 (m, 3H), 6.92 (d, J=7.5 Hz, 1H), 6.79 (s, 1H), 6.73 (dd, J=8.2, 1.8 Hz, 1H), 4.70-4.62 (m, 1H), 4.47 (s, 4H), 4.40 (s, 2H), 2.95 (m, 4H).

### Step 7: M-24

To a solution of M-1 (3.77 g) in THF (40 mL) cooled in an ice bath was added LiHMDS (15.13 mL, 1 M in THF). After stirring at rt for 20 min, M-23 (1.1 g) was added. After stirring at rt overnight, the reaction mixture was quenched with saturated NH₄Cl aqueous solution (50 mL) and extracted with EA (50 mL×3). The EA extract was concentrated and the residue was purified by SGCC eluted with 0 to 100% EA in PE to provide crude M-24 (1.6 g).

### Step 8: M-25

To a solution of M-24 (1.6 g) in THF (20 mL) was added H₂O₂ (1.43 mL, 30%) and LiOH (12.9 mL, aq. 1M). After stirring at rt overnight, saturated Na₂SO₃ aqueous solution (50 mL) was added and the mixture was then acidified by 2 N HCl and extracted with EA (30 mL×3). The EA extract was concentrated to obtain crude M-25.

### Step 9: M-26

To a solution of M-25 in THF (20 mL) was added tert-butyl N,N′-diisopropylcarbamimidate (1.9 g). After stirring at rt overnight, the solution was filtered and the filtrate was concentrated in vacuum. The residue was purified by SGCC eluted with 0 to 50% EA in PE to provide M-26 (300 mg).

### Step 10: T-3

M-26 (300 mg) was treated with HCl 1,4-dioxane (4 mL, 4N). After stirring at rt overnight, the solution was concentrated in vacuum and the residue was purified by Pre-HPLC (flow rate: 15 mL/min; gradient: 0-15 min, 5%-30% acetonitrile/water) to yield 82 mg of T-3 as a white solid.

¹H NMR (400 MHz, D₂O) δ 7.24-6.89 (m, 9H), 6.69 (d, J=7.4 Hz, 1H), 6.54-6.37 (m, 2H), 3.58-3.48 (m, 1H), 3.36-2.45 (m, 22H), 2.27 (m, 6H), 2.06-1.88 (m, 3H), 1.70-1.48 (m, 3H).

MS ESI: m/z=712, [M+1]⁺.

### Example T-4

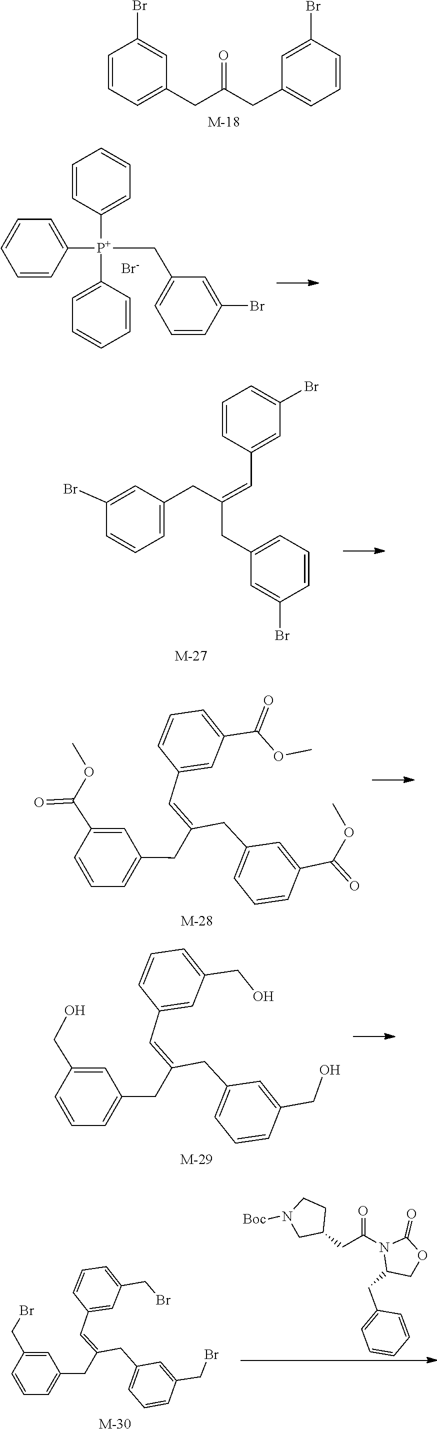

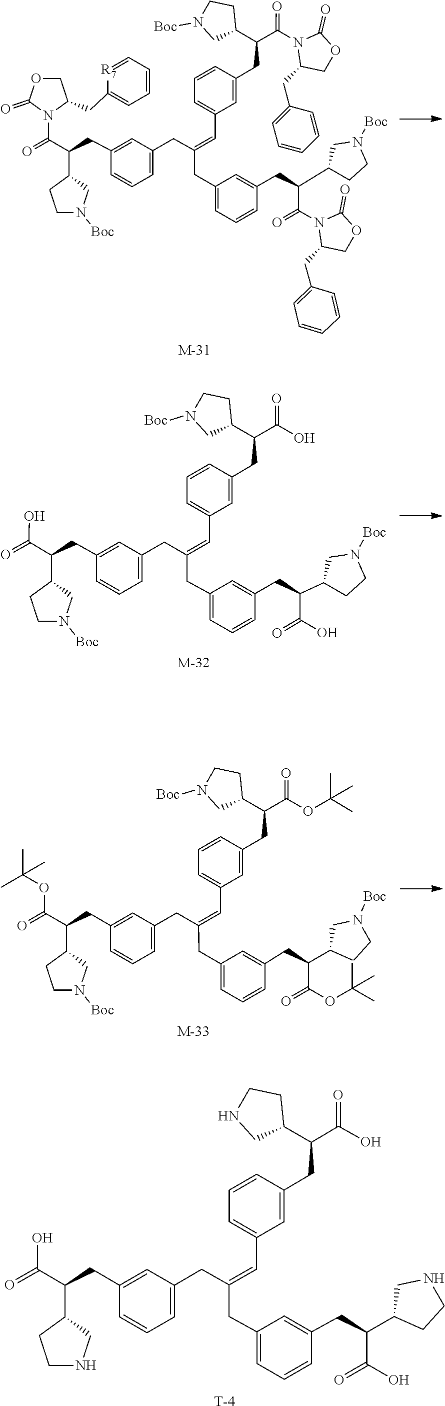

### Step 1: M-27

To a solution of (3-bromobenzyl)triphenylphosphonium bromide (4.17 g) in THF (30 mL) was added t-BuOK (914 mg). After stirring at rt for 1 h, M-18 (2 g) was added. After stirring at 60 °C for 19 h, the reaction mixture was quenched with saturated NH₄Cl aqueous solution (50 mL) and extracted with EA (50 mL×3). The EA extract was concentrated and the residue was purified by SGCC eluted with 0 to 20% EA in PE to provide crude oily M-27 (2.49 g).

¹H NMR (400 MHz, CDCl₃) δ 7.45-7.27 (m, 6H), 7.25-7.13 (m, 4H), 7.11-6.96 (m, 2H), 6.50 (s, 1H), 3.48 (s, 2H), 3.33 (s, 2H).

### Step 2 to 8: T-4

Starting with M-27, T-4 was prepared by using the same conditions described in Step 4 to 10 for T-3.

¹H NMR (400 MHz, D₂O) δ 7.16 (m, 3H), 7.10-7.00 (m, 5H), 6.94 (m, 4H), 6.46 (s, 1H), 3.41 (s, 2H), 3.36-3.18 (m, 8H), 3.17-3.04 (m, 3H), 2.81-2.64 (m, 6H), 2.66-2.53 (m, 3H), 2.45-2.22 (m, 6H), 2.09-1.93 (m, 3H), 1.74-1.52 (m, 3H).

MS ESI: m/z=708, [M+1]⁺.

### Example T9

### Method A

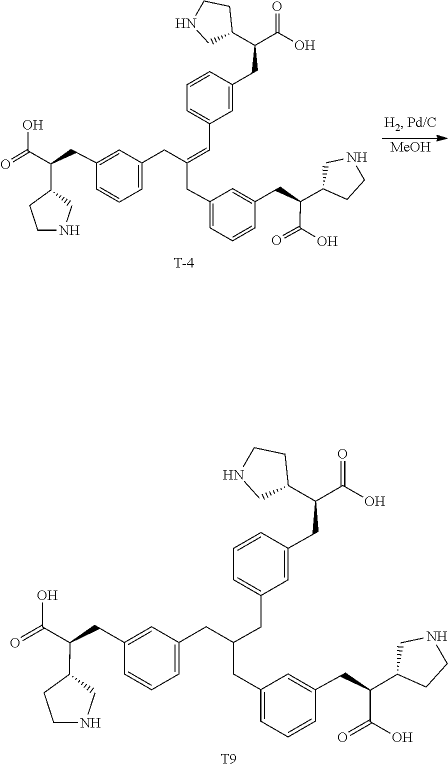

A solution of T4 (5 mg) in 5 mL of MeOH was added 5 mg of Pd/C (10% by weight) and then stirred under H₂ atmosphere (balloon pressure) at rt for 24 h. The mixture was then filtered through a pad of celite and concentrated to give T9 as a white solid (5 mg).

MS ESI: m/z=710 [M+1]⁺.

### Example T9.3HCl

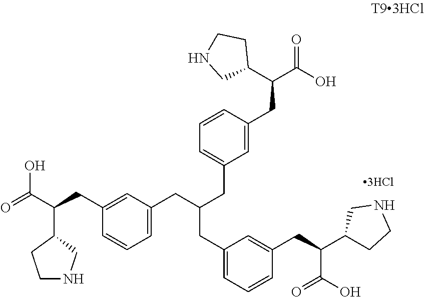 [SMILES: O=C(O)[C@@H](Cc1cccc(CC(Cc2cccc(C[C@H](C(=O)O)[C@H]3CCNC3)c2)Cc2cccc(C[C@H](C(=O)O)[C@H]3CCNC3)c2)c1)[C@H]1CCNC1]

### Method B

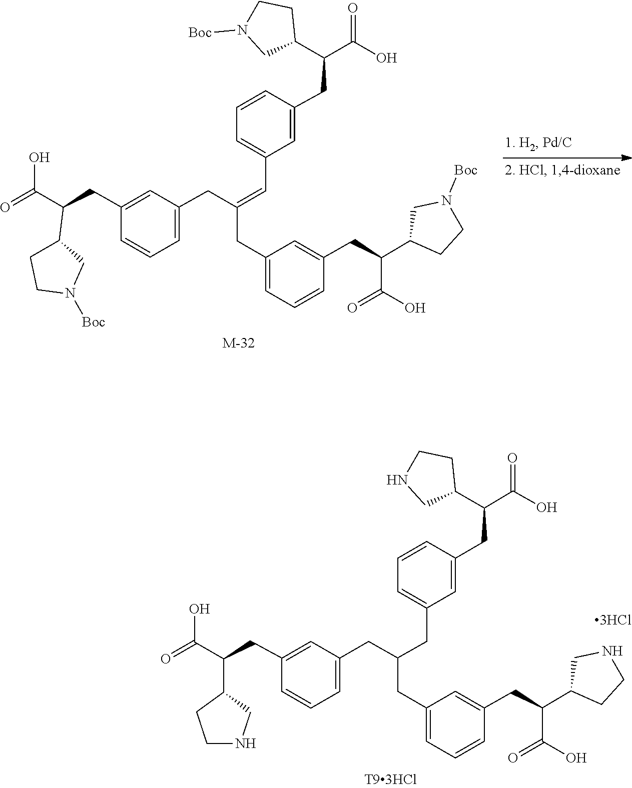 [SMILES: CC(C)(C)OC(=O)N1CC[C@H]([C@H](Cc2cccc(C=C(Cc3cccc(C[C@H](C(=O)O)[C@H]4CCN(C(=O)OC(C)(C)C)C4)c3)Cc3cccc(C[C@H](C(=O)O)[C@H]4CCN(C(=O)OC(C)(C)C)C4)c3)c2)C(=O)O)C1 ; "M-32" ; O=C(O)[C@@H](Cc1cccc(CC(Cc2cccc(C[C@H](C(=O)O)[C@H]3CCNC3)c2)Cc2cccc(C[C@H](C(=O)O)[C@H]3CCNC3)c2)c1)[C@H]1CCNC1]

### Step 1

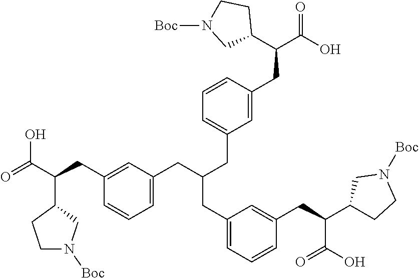 [SMILES: CC(C)(C)OC(=O)N1CC[C@H]([C@H](Cc2cccc(CC(Cc3cccc(C[C@H](C(=O)O)[C@H]4CCN(C(=O)OC(C)(C)C)C4)c3)Cc3cccc(C[C@H](C(=O)O)[C@H]4CCN(C(=O)OC(C)(C)C)C4)c3)c2)C(=O)O)C1]

A mixture of M-32 (22.3 mg) in 6 mL EA and 23.4 mg Pd/C (10%, wet 55%) was stirred overnight at rt under a balloon pressure of hydrogen. The mixture was then filtered. The filtrate was concentrated to give the product as a white solid (24 mg).

### Step 2

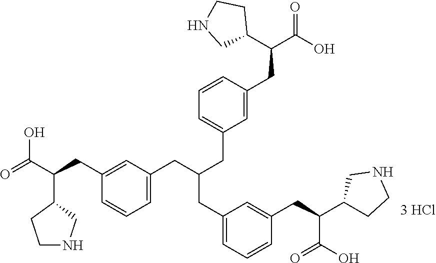 [SMILES: O=C(O)[C@@H](Cc1cccc(CC(Cc2cccc(C[C@H](C(=O)O)[C@H]3CCNC3)c2)Cc2cccc(C[C@H](C(=O)O)[C@H]3CCNC3)c2)c1)[C@H]1CCNC1]

To a solution of the product of the Step 1 (38 mg) in THF (1 mL) was added 2 mL 4 M HCl (1,4-dioxane). After stirring at rt for 16 h, the white solid was abstained by decanting the solvent. The solid was then washed with MTBE (2 mL) and dried under vacuum to provide the hydrochloride of the title product (26.5 mg) as a white solid.

MS ESI: m/z=710 [M+1]⁺.

### Example T19

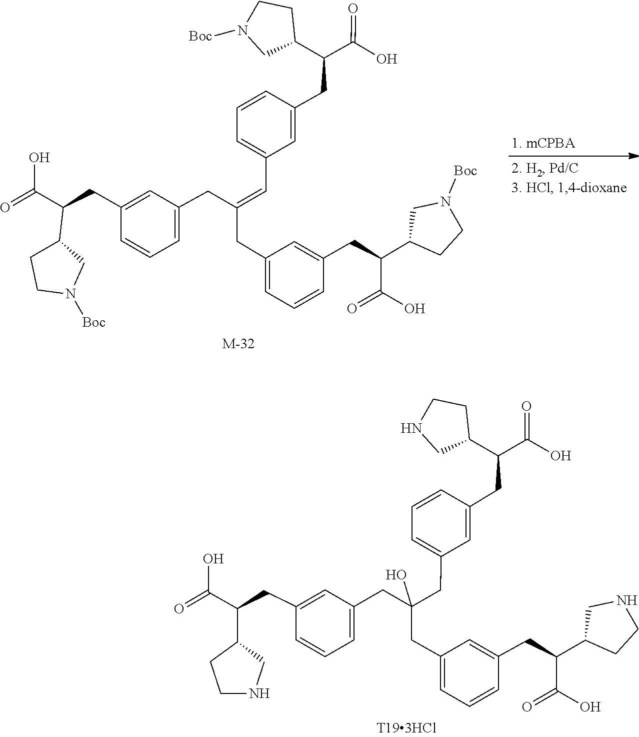 [SMILES: CC(C)(C)OC(=O)N1CC[C@H]([C@H](Cc2cccc(C=C(Cc3cccc(C[C@H](C(=O)O)[C@H]4CCN(C(=O)OC(C)(C)C)C4)c3)Cc3cccc(C[C@H](C(=O)O)[C@H]4CCN(C(=O)OC(C)(C)C)C4)c3)c2)C(=O)O)C1 ; "M-32" ; O=C(O)[C@@H](Cc1cccc(CC(O)(Cc2cccc(C[C@H](C(=O)O)[C@H]3CCNC3)c2)Cc2cccc(C[C@H](C(=O)O)[C@H]3CCNC3)c2)c1)[C@H]1CCNC1]

### Step 1 (2S,2'S)-3,3′-(((3-(3-((S)-2-((R)-1-(tert-butoxycarbonyl)pyrrolidin-3-yl)-2-carboxyethyl)phenyl)oxirane-2,2-diyl)bis(methylene))bis(3,1-phenylene))bis(2-((R)-1-(tert-butoxycarbonyl)pyrrolidin-3-yl)propanoic acid)

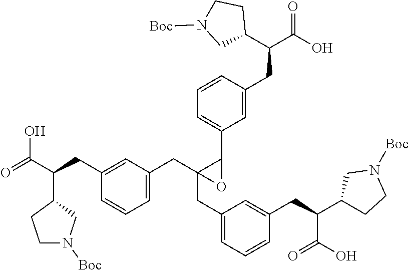 [SMILES: CC(C)(C)OC(=O)N1CC[C@H]([C@H](Cc2cccc(CC3(Cc4cccc(C[C@H](C(=O)O)[C@H]5CCN(C(=O)OC(C)(C)C)C5)c4)OC3c3cccc(C[C@H](C(=O)O)[C@H]4CCN(C(=O)OC(C)(C)C)C4)c3)c2)C(=O)O)C1]

A mixture of M-32 (0.3218 g) in 4 mL EA and 0.1701 g mCPBA (85%) was stirred overnight at rt. The mixture was concentrated. The residue was purified by reverse phase column chromatography (C-18, grain size: 40 m, pore size distribution: 120 Å) eluted with H₂O/ACN (5% to 85%), and lyophilized to afford the title compound as a white solid (0.256 g).

MS ESI: m/z=924.3 [M+1−100]⁺.

### Step 2

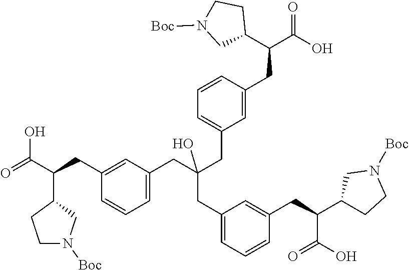 [SMILES: CC(C)(C)OC(=O)N1CC[C@H]([C@H](Cc2cccc(CC(O)(Cc3cccc(C[C@H](C(=O)O)[C@H]4CCN(C(=O)OC(C)(C)C)C4)c3)Cc3cccc(C[C@H](C(=O)O)[C@H]4CCN(C(=O)OC(C)(C)C)C4)c3)c2)C(=O)O)C1]

A mixture of the product of Step 1 (0.20 g) in 10 mL EA and 0.21 g Pd/C (10%, wet 55%) was stirred overnight at rt under a balloon pressure of hydrogen. The mixture was then filtered. The filtrate was concentrated to give the product as a white solid (0.16 g).

MS ESI: m/z=926.3 [M+1-100]⁺.

### Step 3

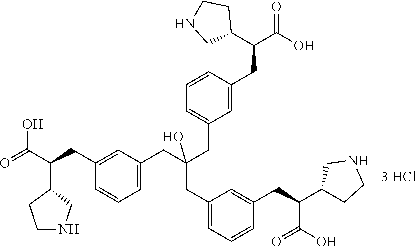 [SMILES: O=C(O)[C@@H](Cc1cccc(CC(O)(Cc2cccc(C[C@H](C(=O)O)[C@H]3CCNC3)c2)Cc2cccc(C[C@H](C(=O)O)[C@H]3CCNC3)c2)c1)[C@H]1CCNC1]

To a solution of the product of the Step 2 (50.0 mg) in THF (1 mL) was added 2 mL 4 M HCl (1,4-dioxane). After left stand at rt for 16 h, the white solid was abstained by decanting the solvent. The solid was then washed with MTBE (2 mL) and dried under vacuum to provide the hydrochloride of the title product (26.2 mg) as a white solid.

MS ESI: m/z=724.4 [M-1]⁻.

### Example T20

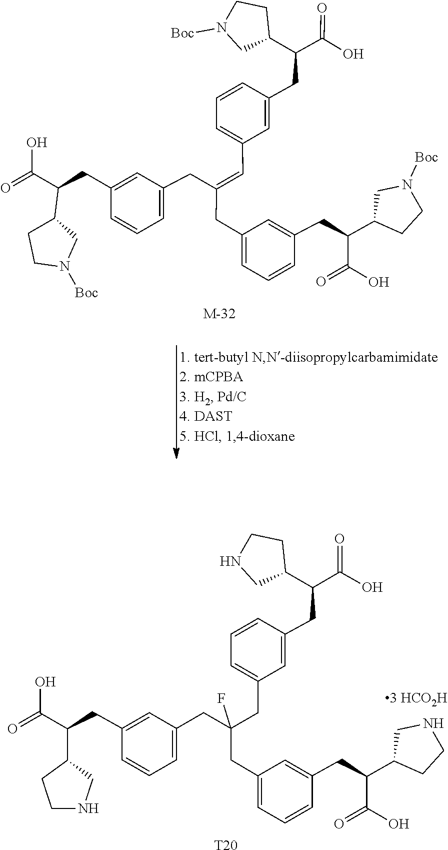 [SMILES: CC(C)(C)OC(=O)N1CC[C@H]([C@H](Cc2cccc(C=C(Cc3cccc(C[C@H](C(=O)O)[C@H]4CCN(C(=O)OC(C)(C)C)C4)c3)Cc3cccc(C[C@H](C(=O)O)[C@H]4CCN(C(=O)OC(C)(C)C)C4)c3)c2)C(=O)O)C1 ; "M-32" ; O=C(O)[C@@H](Cc1cccc(CC(F)(Cc2cccc(C[C@H](C(=O)O)[C@H]3CCNC3)c2)Cc2cccc(C[C@H](C(=O)O)[C@H]3CCNC3)c2)c1)[C@H]1CCNC1 ; "T20"]

### Step 1

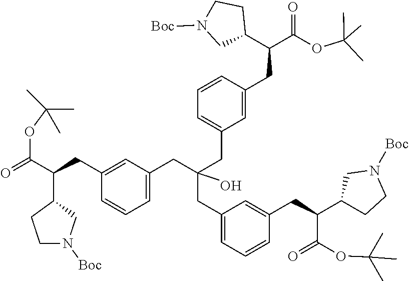 [SMILES: CC(C)(C)OC(=O)[C@@H](Cc1cccc(CC(O)(Cc2cccc(C[C@H](C(=O)OC(C)(C)C)[C@H]3CCN(C(=O)OC(C)(C)C)C3)c2)Cc2cccc(C[C@H](C(=O)OC(C)(C)C)[C@H]3CCN(C(=O)OC(C)(C)C)C3)c2)c1)[C@H]1CCN(C(=O)OC(C)(C)C)C1]

Starting with M-32, the title compound was prepared by using the same conditions described in Step 2 for T21, Step 1 and 2 for T19.

### Step 2

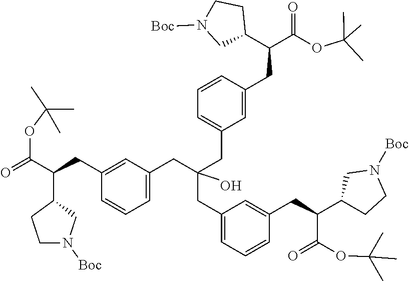 [SMILES: CC(C)(C)OC(=O)[C@@H](Cc1cccc(CC(O)(Cc2cccc(C[C@H](C(=O)OC(C)(C)C)[C@H]3CCN(C(=O)OC(C)(C)C)C3)c2)Cc2cccc(C[C@H](C(=O)OC(C)(C)C)[C@H]3CCN(C(=O)OC(C)(C)C)C3)c2)c1)[C@H]1CCN(C(=O)OC(C)(C)C)C1]

To a solution of the product of Step 1 (0.15 g) in dry DCM (5 mL) cooled at −78 °C was added DAST (0.15 mL). The reaction mixture was stirred for 2 h at rt. The reaction was quenched with aq. NaHCO₃ (5 mL) and extracted with 20 mL of EA. The organic layer was washed with brine, dried over Na₂SO₄, and concentrated. The residue was purified by SGCC eluted with PE/EA (0% to 40%) to afford 15.8 mg the title compound as a white solid.

### Step 3

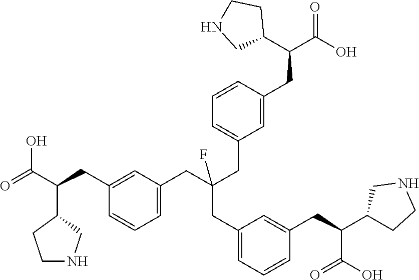 [SMILES: O=C(O)[C@@H](Cc1cccc(CC(F)(Cc2cccc(C[C@H](C(=O)O)[C@H]3CCNC3)c2)Cc2cccc(C[C@H](C(=O)O)[C@H]3CCNC3)c2)c1)[C@H]1CCNC1]

Starting with the product of Step 2, the title compound T-20 was prepared by using the same conditions described in Step4 in T21.

MS ESI: m/z=728.2 [M+1]⁺.

### Example T21

### tris(3-((S)-2-Carboxy-2-((R)-pyrrolidin-3-yl)ethyl)benzyl)amine oxide tetratrifluoroacetate

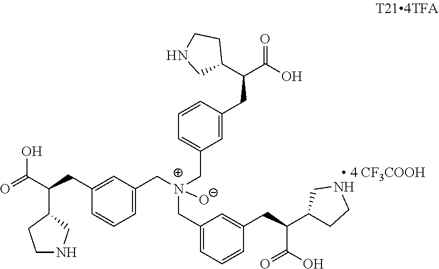 [SMILES: O=C(O)[C@@H](Cc1cccc(C[N+]([O-])(Cc2cccc(C[C@H](C(=O)O)[C@H]3CCNC3)c2)Cc2cccc(C[C@H](C(=O)O)[C@H]3CCNC3)c2)c1)[C@H]1CCNC1]

### Step 1 (2S,2'S,2″S)-3,3′,3″-((nitrilotris(methylene))tris(benzene-3,1-diyl))tris(2-((R)-1-(tert-butoxycarbonyl)pyrrolidin-3-yl)propanoic acid)

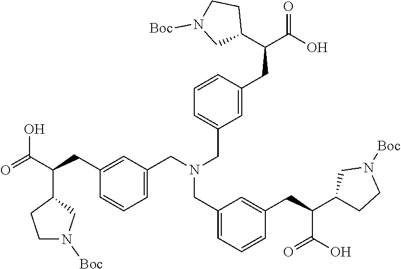 [SMILES: CC(C)(C)OC(=O)N1CC[C@H]([C@H](Cc2cccc(CN(Cc3cccc(C[C@H](C(=O)O)[C@H]4CCN(C(=O)OC(C)(C)C)C4)c3)Cc3cccc(C[C@H](C(=O)O)[C@H]4CCN(C(=O)OC(C)(C)C)C4)c3)c2)C(=O)O)C1]

A mixture of (2S,2'S,2″S)-3,3′,3″-((nitrilotris(methylene))tris(benzene-3,1-diyl))tris(2-((R)-pyrrolidin-3-yl)propanoic acid) (0.29 g), NaOH (0.1461 g) and (Boc)₂O (0.40 mL) in 5 mL THF and 1 mL water was stirred overnight at rt. 1.64 g citric acid and 10 mL EA were added. The aqueous layer was separated. The organic layer was washed with water (10 mL), brine and dried over Na₂SO₄. The organic layer was concentrated to give 0.22 g the crude title compound as a yellow solid.

MS ESI: m/z=1011.4 [M+1]⁺.

### Step 2 tri-tert-butyl 3,3′,3″-((2S,2'S,2″S)-((nitrilotris(methylene))tris(benzene-3,1-diyl))tris(3-(tert-butoxy)-3-oxopropane-1,2-diyl))(3R,3′R,3″R)-tris(pyrrolidine-1-carboxylate)

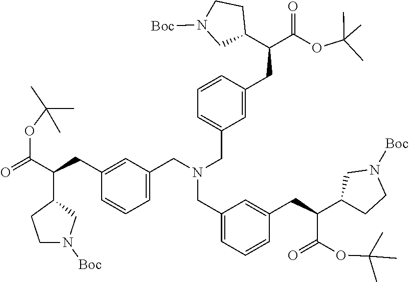 [SMILES: CC(C)(C)OC(=O)[C@@H](Cc1cccc(CN(Cc2cccc(C[C@H](C(=O)OC(C)(C)C)[C@H]3CCN(C(=O)OC(C)(C)C)C3)c2)Cc2cccc(C[C@H](C(=O)OC(C)(C)C)[C@H]3CCN(C(=O)OC(C)(C)C)C3)c2)c1)[C@H]1CCN(C(=O)OC(C)(C)C)C1]

To a mixture of the crude product of the Step 1 (0.22 g) in 5 mL THF was added tert-butyl N,N′-diisopropylcarbamimidate (2.0 mL) in four portions under 65 °C. The reaction mixture was stirred overnight at 65 °C. The solid was removed by filtration and the filtrate was washed with aq. NaHCO₃ and dried over Na₂SO₄. The organic layer was concentrated and the residue was suspended in 10 mL MTBE. The solid was removed by filtration and the filtrate was concentrated to give 0.234 g the crude title compound as a yellow oil.

MS ESI: m/z=1079.5 [M+1-100]⁺.

### Step 3 tris(3-((S)-3-(tert-butoxy)-2-((R)-1-(tert-butoxycarbonyl)pyrrolidin-3-yl)-3-oxopropyl)benzyl)amine oxide

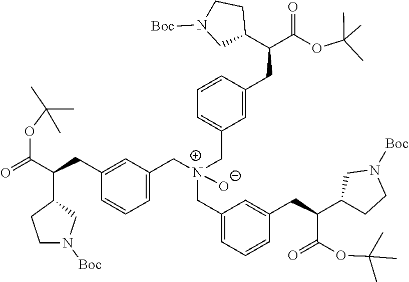 [SMILES: CC(C)(C)OC(=O)[C@@H](Cc1cccc(C[N+]([O-])(Cc2cccc(C[C@H](C(=O)OC(C)(C)C)[C@H]3CCN(C(=O)OC(C)(C)C)C3)c2)Cc2cccc(C[C@H](C(=O)OC(C)(C)C)[C@H]3CCN(C(=O)OC(C)(C)C)C3)c2)c1)[C@H]1CCN(C(=O)OC(C)(C)C)C1]

A mixture of the product of the Step 2 (0.23 g) in 5 mL DCM was added MCPBA (0.0690 g) and NaHCO₃ (0.0311 g) at rt. The reaction mixture was stirred overnight at rt and then concentrated. The residue was purified by SGCC eluted with DCM/MeOH (0% to 10%) to give 0.076 g the title compound as white solid.

MS ESI: m/z=1195.4 [M+1]⁺.

### Step 4 tris(3-((S)-2-carboxy-2-((R)-pyrrolidin-3-yl)ethyl)benzyl)amine oxide tetratrifluoroacetate

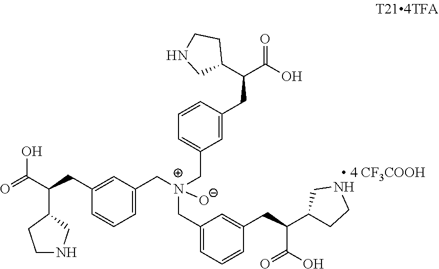 [SMILES: O=C(O)[C@@H](Cc1cccc(C[N+]([O-])(Cc2cccc(C[C@H](C(=O)O)[C@H]3CCNC3)c2)Cc2cccc(C[C@H](C(=O)O)[C@H]3CCNC3)c2)c1)[C@H]1CCNC1]

To a solution of the product of the Step 3 (20.0 mg) in THF (0.5 mL) was added 0.5 mL of 4 M HCl (1,4-dioxane). After 16 h, the solvent was decanted and the remaining solid was purified by prep-HPLC (WATERS XBridge C18, 19×150 mm, 5 um) eluted with ACN/water (0.1% TFA, 15 mL/min, 10% to 20% in 9 min) to provide the title product (9.65 mg) as a white solid.

MS ESI: m/z=727.3 [M+1]⁺.

### Example T22

### N,N-bis(3-((S)-2-carboxy-2-((R)-pyrrolidin-3-yl)ethyl)benzyl)-2-(3-((S)-2-carboxy-2-((R)-pyrrolidin-3-yl)ethyl)phenoxy)ethan-1-amine oxide hydochloride

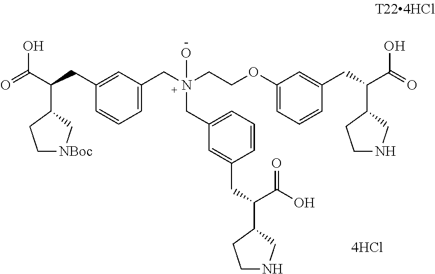

### Step 1 di-tert-butyl 3,3′-((2S,2'S)-((((2-(3-((S)-3-(tert-butoxy)-2-((R)-1-(tert-butoxycarbonyl)pyrrolidin-3-yl)-3-oxopropyl)phenoxy)ethyl)azanediyl)bis(methylene))bis(3,1-phenylene))bis(3-(tert-butoxy)-3-oxopropane-1,2-diyl))(3R,3′R)-bis(pyrrolidine-1-carboxylate)

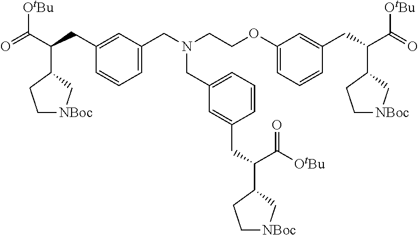 [SMILES: CC(C)(C)OC(=O)[C@@H](Cc1cccc(CN(CCOc2cccc(C[C@H](C(=O)OC(C)(C)C)[C@H]3CCN(C(=O)OC(C)(C)C)C3)c2)Cc2cccc(C[C@H](C(=O)OC(C)(C)C)[C@H]3CCN(C(=O)OC(C)(C)C)C3)c2)c1)[C@H]1CCN(C(=O)OC(C)(C)C)C1]

A mixture of tert-butyl (R)-3-((S)-3-(3-(2-aminoethoxy)phenyl)-1-(tert-butoxy)-1-oxopropan-2-yl)pyrrolidine-1-carboxylate (0.1588 g), tert-butyl (R)-3-((S)-1-(tert-butoxy)-3-(3-formylphenyl)-1-oxopropan-2-yl)pyrrolidine-1-carboxylate (0.4134 g), HOAc (0.021 mL) and NaBH₃CN (0.1558 g) in 6 mL of MeOH was stirred overnight at 65 °C. To the reaction mixture, 1 mL of aq. saturated K₂CO₃ solution was then added. The reaction mixture was concentrated and treated with Na₂SO₄ and 5 mL DCM were added. The DCM solution was concentrated and the residue was purified by SGCC eluted with PE/EA (0% to 30%) to give 0.387 g the title compound as an oil.

### Step 2 N,N-bis(3-((S)-3-(tert-butoxy)-2-((R)-1-(tert-butoxycarbonyl)pyrrolidin-3-yl)-3-oxopropyl)benzyl)-2-(3-((S)-3-(tert-butoxy)-2-((R)-1-(tert-butoxycarbonyl)pyrrolidin-3-yl)-3-oxopropyl)phenoxy)ethan-1-amine oxide

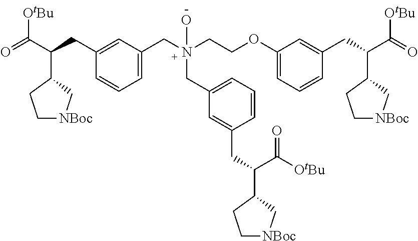

To a solution of the product of Step 1 (0.10 g) in 5 mL DCM was added MCPBA (0.0383 g) at rt. After stirring for 2 h at rt. The reaction mixture was concentrated and the residue was purified by SGCC eluted with DCM/MeOH (0% to 10%) to give 0.076 g the title compound as an oil.

MS ESI: m/z=563.3 [(M+2-100)/2]⁺.

### Step 3 N,N-bis(3-((S)-2-carboxy-2-((R)-pyrrolidin-3-yl)ethyl)benzyl)-2-(3-((S)-2-carboxy-2-((R)-pyrrolidin-3-yl)ethyl)phenoxy)ethan-1-amine oxide hydrochloride

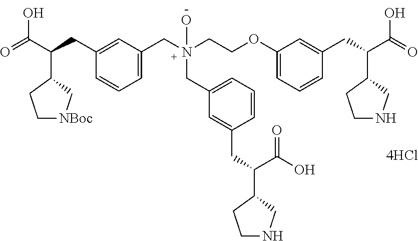

To a solution of the title compound of Step 2 (17.5 mg) in THF (0.5 mL) was added 1 mL 4 M HCl (1,4-dioxane). After stirring for 16 h, a white solid was formed. The solvent was carefully decanted and the white solid was dried under vacuum to give the title product (3.4 mg).

MS ESI: m/z=757.3 [M+1]⁺.

### Cellular Assay for Inhibition of Lp(a) Assembly

Protocol 2 (performed at Pharmaron Beijing Co. Ltd)
HepG2 cells (endogenously express ApoB, ATCC, cat No. HB-8065);
HEK293 cells that stably express and secrete recombinant human 16K Apo(a) protein containing 17 Kringle IV repeats.
   The assembly assay was conducted in 96-well polypropylene plates by combining HepG2 and apo(a)-HEK293-conditioned media in the presence of a range of compound concentrations. After a two-hour incubation period at 37 °C, the reaction was stopped by the addition of 6-aminocaproic acid. Lp(a) was detected using a sandwich enzyme-linked immunosorbent assay (ELISA), with an anti-apo (a) antibody (Abcam ab242565) and a HRP anti-ApoB detection antibody (Abcam ab27622).

TABLE 1
Lp(a) Assembly Inhibitory Activity

| | Tested | | | |
|---|---|---|---|---|
| | concentration | % | IC₅₀ | IC₉₀ |
| Example | (nM) | inhibition | (nM) | (nM) |
| Muvalaplin | | | 0.16 | 0.69 |
| Muvalaplin | 0.12 | 42.71 | | |
| T-3 | 1.6 | 92.4 | | |
| T-4 | 1.6 | 93.4 | | |
| T9•3HCl | | | <0.16 | <0.8 |
| T9•3HCl | 0.16 | 78.68 | | |
| T20 | | | 0.45 | 2.55 |
| T21•4TFA | | | 0.18 | 0.56 |
| T22•4TFA | | | 0.23 | 0.71 |

The assay data showed that compounds in the invention possess potent Lp(a) assembly inhibitory activity.

### Pharmacokinetics Parameters

### 1. Mouse Pharmacokinetics Study Protocol

Male ICR mice were supplied by Charles River. The intragastric (p.o.) administration was performed with a dosage of 5 mg/kg or 10 mg/kg. Preparation: the compound was dissolved in a formulation made of 0.25% tween 80, 1% HEC, and 98.75% water. The mixture was shaken by vortex and/or ultrasound until the compound was uniformly distributed with a small particle size, or the compound was dissolved as a clear solution. Blood samples were taken at 15 minutes, 30 minutes, 1 hour, 2 hours, 4 hours, 6 hours, 8 hours, and 24 hours after administration.

The intravenous administration was performed with a dosage of 1 mg/kg. Preparation: 10% DMA, 10% solutol, and 80% physiological saline or 100% physiological saline was added to the compound, and mixed well. Blood samples were taken at 5 minutes, 15 minutes, 30 minutes, 1 hour, 2 hours, 4 hours, 8 hours, and 24 hours after administration.

The samples were processed and analyzed using a method of liquid chromatography tandem mass spectrometry (LC-MS/MS).

### Mouse PK Parameters

| | Dosage | Tₘₐₓ | Cₘₐₓ | T_{1/2} | AUC_{0-24 h} |
|---|---|---|---|---|---|
| Example | n = 2 | hr | ng/mL | hr | hr · ng/mL |
| T9 · 3HCl | 10 mg/kg | 4.0 | 570 | 10.5 | 8052 |

pharmacokinetics study protocol

Male SD rats were supplied by Charles River. The intragastric (p.o.) administration was performed with a dosage of 5 mg/kg. Preparation: the compound was dissolved in a formulation made of 0.25% tween 80, 1% HMPC, and 98.75% water. The mixture was shaken by vortex and/or ultrasound until the compound was uniformly distributed with a small particle size, or the compound was dissolved as a clear solution. Blood samples were taken at 15 minutes, 30 minutes, 1 hour, 2 hours, 4 hours, 6 hours, 8 hours, and 24 hours after administration.

The intravenous administration was performed with a dosage of 1 mg/kg. Preparation: 10% DMA, 10% solutol, and 80% physiological saline or 100% physiological saline were added to dissolve the compound, and mixed well. Blood samples were taken at 5 minutes, 15 minutes, 30 minutes, 1 hour, 2 hours, 4 hours, 8 hours, and 24 hours after administration.

The samples were processed analyzed using a method of liquid chromatography tandem mass spectrometry (LC-MS/MS).

### Rat PK Parameters

LP(a)

| | Dosage | | Tₘₐₓ | Cₘₐₓ | T_{1/2} | AUC_{0-24 h} |
|---|---|---|---|---|---|---|
| Example | n = 2 | | hr | ng/mL | hr | hr · ng/mL |
| T9•3HCl | 5 | mg/kg | 3.0 | 56 | 18 | 606 |
| T9•3HCl | 20 | mg/kg | 4.0 | 669 | 12 | 10027 |

transgenic mouse studies (performed at Pharmaron Beijing Co. Ltd)

B6-RCL-hLPA/Alb-cre/Tg(APOB) mice (male, age 6-8 weeks) randomized to 4 treatment groups (n=4 per group) and all mice were prescreened for Lp(a) levels as pretreatment baseline levels. Mice were dosed with Muvalaplin and Example T9-3HCl in vehicle (10 mL/kg, 1% hydroxyethyl cellulose, 0.25% Tween-80, 0.01% antifoam) orally BID for 5 days. Blood samples were collected by tail bleeds at the time before first dose administration, 8 h post first dosing each day and the Lp(a) levels were determined by ELISA.

### LP(a) Reduction on Day 5

| | | |
|---|---|---|
| | | % Inhibition |
| Example | Dosage | of Lp(a) |

| | | | |
|---|---|---|---|
| Muvalaplin | 3 | mg/kg | 14.1 |
| T9•3HCl | 3 | mg/kg | 53.0 |
| Muvalaplin | 10 | mg/kg | 76.8 |
| T9•3HCl | 10 | mg/kg | 83.4 |

Example T9·3HCl unexpectedly exhibited superior Lp(a) reduction to that of Muvalaplin at both dosages of 3 mg/kg and 10 mg/kg.

Muvalaplin (LY3473329) was prepared according the methods published in WO2020247429.

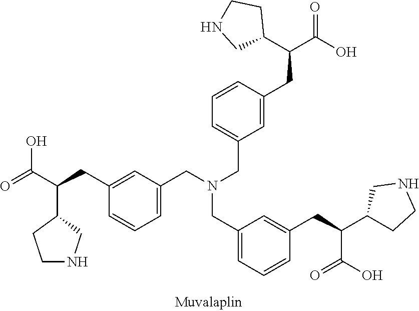 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

In this specification, the invention has been described with reference to its specific embodiments. However, it is evident that various modifications and changes can be made without departing from the spirit and scope of the invention. Therefore, the specification should be considered as illustrative and not restrictive.

1. A compound represented by formula IA or its pharmaceutically acceptable salt: 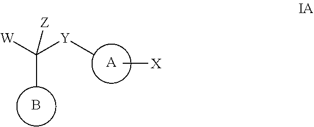
wherein,
W is CO₂H;
X is selected from the following moieties: 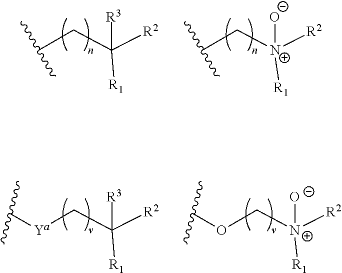
Y^{a} is O, S, CH₂, NH, NC₁₋₆alkyl, NC₃₋₆cycloalkyl, N-phenyl, NC(O)C₁₋₆alkyl, NC(O) C₃₋₆yccloalkyl or NC(O) phenyl; the said alkyl, cycloalkyl and phenyl are unsubstituted or substituted with one or more halogen;
R¹ and R² are independently selected the following moieties: 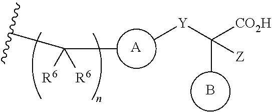 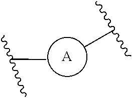
is independently selected from: 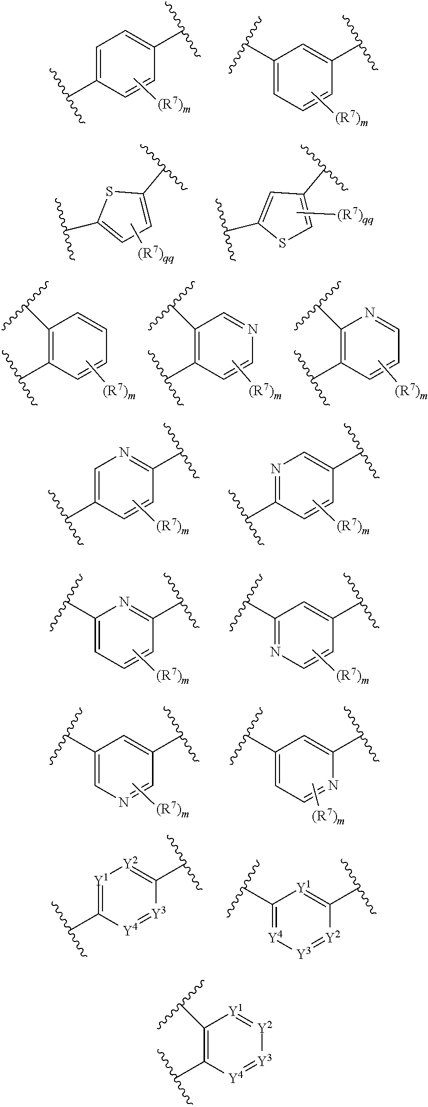
Y¹, Y², Y³ and Y⁴ are independently CR⁷ or N; 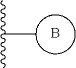
is independently 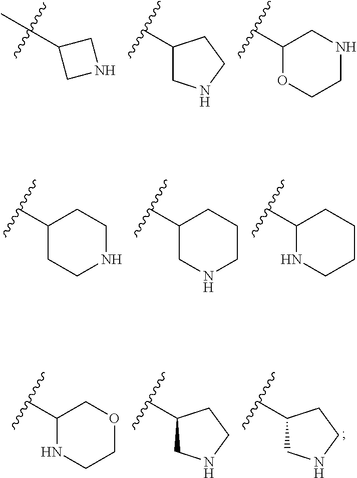
R³ is independently H, -L¹-R^{x}, —(CH₂)ₙOH, —(CH₂)ₙNH₂, —(CH₂)ₙN(R¹⁹) (R²⁰), —(CH₂)ₙC₃₋₆cycloalkyl or —(CH₂)ₙC₆₋₁₀aryl;
L¹ is a chemical bond, —C≡C—, arylene or heteroarylene;
R^{x} is independently H, or —(CH₂)ₙ-halogen;
Z is independently H, halogen, C₁₋₄ alkyl, —OC₁₋₄ alkyl, OH, cyclopropyl, or —CH₂OH;
Y is independently (C(R⁶)₂)_{g};
g is an integer of 0 to 4;
R⁶ is independently selected from hydrogen, halogen, —C₁₋₆alkyl, —C₁₋₆haloalkyl, —C₃₋₁₀heteroalkyl, —C₃₋₆cycloalkyl, —OC₁₋₆alkyl, —OC₁₋₆haloalkyl, —OC₃₋₆cycloalkyl; or two R⁶ can be connected to form a 3 to 10 membered carbocyclic ring or heterocyclic ring in which the heteroatom is O, S or NR^{d};
R⁷ is independently OH, CN, SF₅, halo, C₁₋₆alkyl, C₃₋₁₀cycloalkyl, C₃₋₁₀heteroalkyl, C₃₋₁₀ heterocylcoalkyl, —C₁₋₆haloalkyl, —C₁₋₆alkoxy, N(R¹⁹)₂, —C₃₋₈cycloalkyl, —C₂₋₄alkenyl, C₂₋₄alkynyl, C₂₋₆heterocyclyl, aryl or heteroaryl;
qq is independently integer of 0 to 2;
n is independently an integer of 0 to 6;
m is independently an integer of 0 to 4;
R¹⁹ and R²⁰ are independently selected from hydrogen, C₁₋₆alkyl, C₃₋₁₀heteroalkyl, C₃₋₆cycloalkyl, C₆₋₁₀aryl or a C₁₋₅heteroaryl, C₂₋₆heterocyclyl;
R^{d} is independently hydrogen, acyl, hydroxyl, C₁₋₈hydroxyalkyl, C₁₋₈alkyl, aralkyl, C₁₋₈haloalkyl, C₁₋₈heteroalkyl, C₃₋₁₀cycloalkyl, C₁₋₁₀heterocyclylalkyl, C₁₋₈alkoxy, C(O)NH₂, C(O)NHC₁₋₆alkyl, C(O)NHC₃₋₆cycloalkyl, C(O)N(C₁₋₆alkyl)₂, S(O)₂C₁₋₆alkyl, S(O)₂C₁₋₆cycloalkyl, S(O)₂NH₂, S(O)₂NHC₁₋₆alkyl, S(O)₂NHC₃₋₆cycloalkyl, NHC(O)NH₂, NHC(O)NHC₁₋₆alkyl, NHC(O)NHC₁₋₆cycloalkyl, NHC(O)OC₁₋₆alkyl, C(O)—C₁₋₆cycloalkyl, C(O)C₁₋₆alkylamino, C₁₋₆heteroalkyl, P(O)(C₁₋₆alkyl)₂, heterocyclyl, heterocyclylalkyl, N(C₁₋₈alkyl)₂ or C₁₋₈alkyl-N(C₁₋₈alkyl)₂;
v is an integer of 1 to 6.

2. The compound according to claim 1 represented by formula IB or its pharmaceutically acceptable salt: 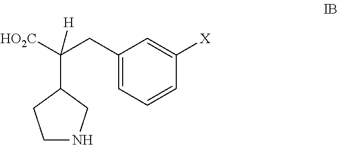
wherein,
X is selected from the following moieties: 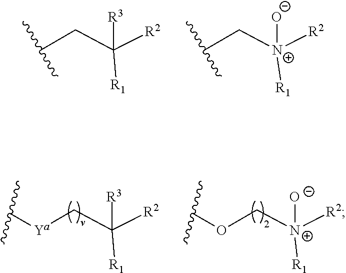
Y^{a} is O, S, CH₂, NH or NC₁₋₆alkyl;
R¹ and R² are 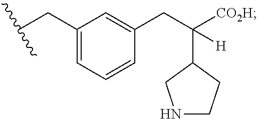 [SMILES: *Cc1cccc(CC(C(=O)O)C2CCNC2)c1]
R³ is independently selected from H, halogen, CN, -L¹-R^{x}, —(CH₂)ₙOH, —(CH₂)ₙNH₂, —(CH₂)ₙN (R¹⁹) (R²⁰), —(CH₂)ₙC₃₋₆cycloalkyl, or —(CH₂)ₙC₆₋₁₀aryl.
L¹ is selected from a chemical bond, —C≡C—, arylene or heteroarylene;
R^{x} is independently selected from H, C₁₋₆alkyl, or —(CH₂)ₙ-halogen;
n is independently an integer of 0 to 6;
v is an integer of 1 or 2;
R¹⁹ and R²⁰ are independently selected from H, C₁₋₆alkyl, C₃₋₁₀heteroalkyl, C₃₋₆cycloalkyl, C₆₋₁₀aryl or heteroaryl, or C₂₋₆heterocycle.

3. The compound according to claim 2 represented by formula XXXXII or its pharmaceutically acceptable salt: 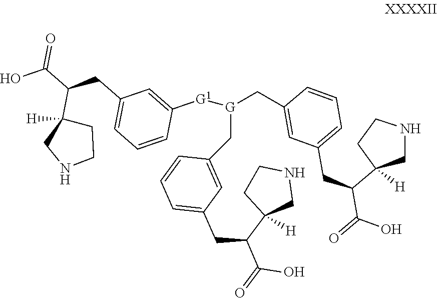 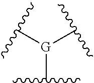
is selected from the following moieties: 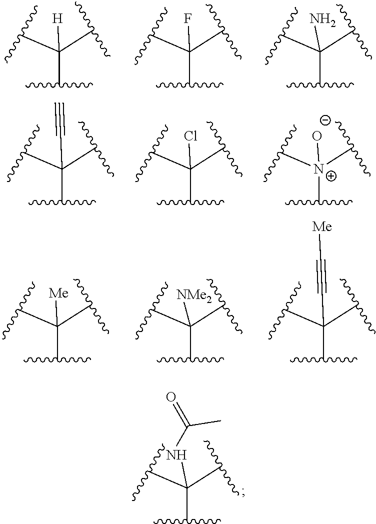
G¹ is —O—CH₂—, —OCH₂CH₂—, —CH₂CH₂—, —CH₂CH₂CH₂— or —CH₂—.

4. The compound according to claim 3 selected from:

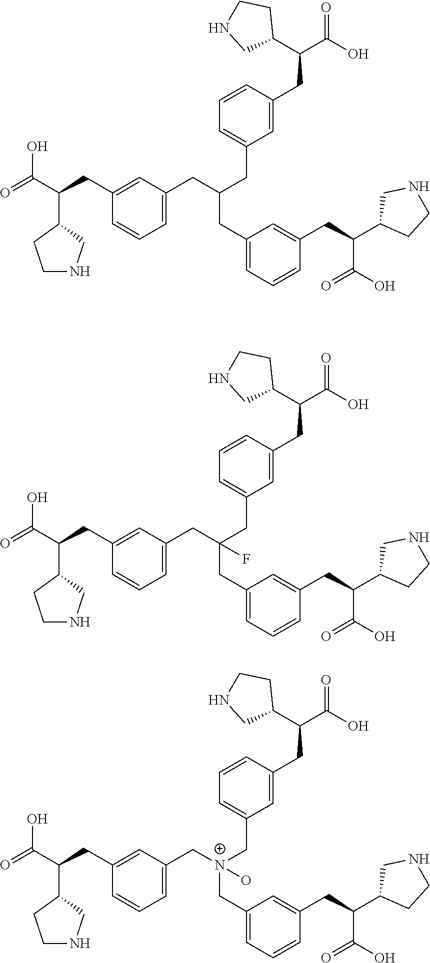 [SMILES: O=C(O)[C@@H](Cc1cccc(CC(Cc2cccc(C[C@H](C(=O)O)[C@H]3CCNC3)c2)Cc2cccc(C[C@H](C(=O)O)[C@H]3CCNC3)c2)c1)[C@H]1CCNC1 ; O=C(O)[C@@H](Cc1cccc(CC(F)(Cc2cccc(C[C@H](C(=O)O)[C@H]3CCNC3)c2)Cc2cccc(C[C@H](C(=O)O)[C@H]3CCNC3)c2)c1)[C@H]1CCNC1 ; O=C(O)[C@@H](Cc1cccc(C[N+](O)(Cc2cccc(C[C@H](C(=O)O)[C@H]3CCNC3)c2)Cc2cccc(C[C@H](C(=O)O)[C@H]3CCNC3)c2)c1)[C@H]1CCNC1]

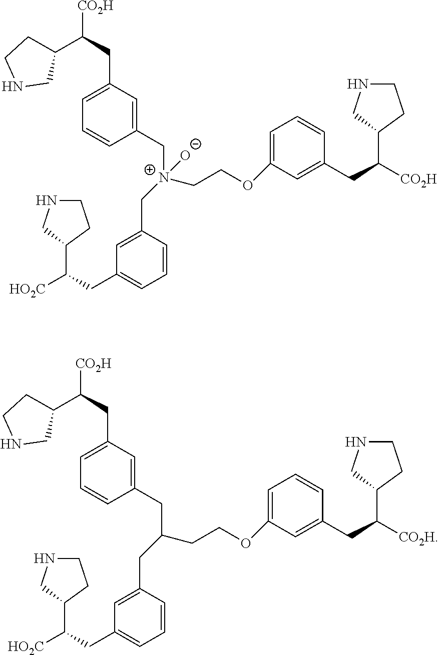 [SMILES: O=C(O)[C@@H](Cc1cccc(C[N+]([O-])(CCOc2cccc(C[C@H](C(=O)O)[C@H]3CCNC3)c2)Cc2cccc(C[C@H](C(=O)O)[C@H]3CCNC3)c2)c1)[C@H]1CCNC1 ; O=C(O)[C@@H](Cc1cccc(CC(CCOc2cccc(C[C@H](C(=O)O)[C@H]3CCNC3)c2)Cc2cccc(C[C@H](C(=O)O)[C@H]3CCNC3)c2)c1)[C@H]1CCNC1]

5. An isotope-labeled analog of a compound of claim 4 or its pharmaceutically accepted salt, in which the isotope is deuterium.

6. Use of the compounds of claim 4 or its pharmaceutically accepted salt for the treatment or prevention of diseases caused by high plasma levels of Lp(a), which include atherosclerotic cardiovascular diseases (including peripheral vascular disease, coronary heart disease, and ischemic stroke), coronary artery stenosis, aortic valve stenosis, heart failure, and atrial fibrillation.
